(19) United States
(12) Patent Application Publication
(10) Pub. No.: US 2006/0263336 A1
(43) Pub. Date: Nov. 23, 2006

(54) Cell targeting methods and compositions

(76) Inventor: Arnold I Caplan, Cleveland Heights, OH (US)

Correspondence Address: Tarolli Sundheim;Covell & Tummino, Suite 1111, 526 Superior Avenue, Cleveland, OH, 44114-1400, US

(21) Appl. No.: 10/550,590

(22) PCT Filed: Mar. 22, 2004
(86) PCT No.: PCT/US04/08763

§ 371 Date: Jun. 1, 2006

Related U.S. Application Data
(60) Provisional application No. 60/457,151, filed on Mar. 24, 2003.

(51) Int. Cl. A61K 35/12; A61K 38/17; C07K 14/705

(52) U.S. Cl. 424/93.7; 530/350; 514/12; 435/366; 435/325

(57) ABSTRACT

In certain aspects, the invention relates to cell delivery compositions comprising a progenitor cell and a targeting moiety, and methods related thereto. Such compositions and methods may be used, for example, in administering a targeted cell therapy to a subject.

### CROSS-REFERENCE TO RELATED APPLICATIONS

This application claims the benefit of priority of U.S. Provisional Application No. 60/457,151 filed Mar. 24, 2003, the specification of which is incorporated by reference herein in its entirety.

### BACKGROUND

Promises of cures of a wide variety of diseases or tissue injuries by specific replacement of damaged or malfunctional tissues by use of totipotent, pluripotent or multipotent stem cells is on the horizon in clinical practice (see, e.g., Fuchs, et al., 2000, Cell, 100:143-156; Weissman et al., 2000, Cell, 100:157-168; Blau, et al., 2001, Cell, 105:829-841). To transmute a somatic cell into the variety of cell types needed for tissue regeneration and reconstruction in vertebrates is a realistic goal. In fact, tissues that were formerly considered incapable of extensive regeneration, such as brain, spinal cord, and cardiac muscle, now appear to be capable of reconstruction functionally, at least to some extent, by stem cell populations. Stem cells derived from the embryo and from adult tissues have been shown to have extensive potentials for self-renewal and differentiation. However, methods of targeting of stem cells to specific target tissues and their potential value for use in tissue reconstruction procedures require further study. Investigation in these areas may lead to realistic approaches in the future for stem cell therapy in a variety of human diseases, tissue injuries, and other clinical problems.

In addition, efforts in tissue engineering and restorative surgery would be improved by advances in cell targeting technology. For example, current applications of tissue engineering to articular cartilage have focused on manipulating cartilage-forming cells, committed chondrocytes or osteochondral progenitor cells as a source for the tissue regenerated. One of the cornerstones/obstacles in implementing this technology is being able to direct the cells or tissue, engineered in vitro, to the precise in vivo site were repair is needed.

### SUMMARY OF THE INVENTION

Certain aspects of this invention provide compositions and methods for delivering progenitor cells to target tissues. In one aspect, the invention provides cell delivery compositions comprising a progenitor cell and a targeting moiety that binds to a target tissue, wherein the targeting moiety selectively directs the progenitor cell to the target tissue. In another aspect, the invention provides methods of delivering a progenitor cell to a target tissue in a subject. Such methods may include a two-step targeting approach, comprising: a) coating a progenitor cell with a linker; b) contacting the coated progenitor cell with a targeting moiety that binds to the linker and can then bind to the target tissue; and c) administering the progenitor cell complexed with the targeting moiety to a subject. Optionally, such methods may include a one-step targeting approach, comprising: a) coating the progenitor cell with a targeting moiety that binds to a target tissue and the progenitor cell; and b) administering the progenitor cell complexed with the targeting moiety to a subject. In either case, the targeting moiety selectively directs the progenitor cell to the target tissue.

In certain embodiments, the progenitor cell is selected from the group consisting of a totipotent stem cell, pluripotent stem cell, multipotent stem cell, mesenchymal stem cell, neuronal stem cell, hematopoietic stem cell, pancreatic stem cell, cardiac stem cell, embryonic stem cell, embryonic germ cell, neural crest stem cell, kidney stem cell, hepatic stem cell, lung stem cell, hemangioblast cell, and endothelial progenitor cell. Optionally, the progenitor cell is selected from a de-differentiated chondrogenic cell, myogenic cell, osteogenic cell, tendogenic cell, ligamentogenic cell, adipogenic cell, and dermatogenic cell.

In certain embodiments, the progenitor cell is pre-coated with a linker, such as a protein G or protein A. Optionally, the linker is modified with a lipophilic moiety which includes without limitation, a palmitoyl moiety, myristoyl moiety, margaroyl moiety, stearoyl moiety, arachidoyl moiety, acetyl moiety, butytyl moiety, hexanoyl moiety, octanoyl moiety, decanoyl moiety, lauroyl moiety, palmitoleoyl moiety, behenoyl moiety, and lignoceroyl moiety. A preferred lipophilic moiety is a palmitoyl moiety, a myristoyl moiety or a margaroyl moiety.

In other embodiments, the progenitor cell is directly linked to the targeting moiety. Optionally, the targeting moiety is modified with a lipophilic moiety which includes without limitation, a palmitoyl moiety, myristoyl moiety, margaroyl moiety, stearoyl moiety, arachidoyl moiety, acetyl moiety, butytyl moiety, hexanoyl moiety, octanoyl moiety, decanoyl moiety, lauroyl moiety, palmitoleoyl moiety, behenoyl moiety, and lignoceroyl moiety. A preferred lipophilic moiety is a palmitoyl moiety, a myristoyl moiety or a margaroyl moiety.

In certain embodiments, the progenitor cell expresses a cell surface marker or an extracellular matrix molecule, for example, CD4, CD8, CD10, CD30, CD33, CD34, CD38, CD45, CD133, CD146, fetal liver kinase-1 (Flk1), C-Kit, Lin, Mac-1, Sca-1, Stro-1, Thy-1, collagen types II or IV, O1, O4, N-CAM, or stage-specific embryonic antigen (SSEA).

In certain embodiments, the targeting moiety comprises a component of a specific binding pair. Preferably, the targeting moiety interacts with an epitope intrinsic to the target tissue. Optionally, the epitope may be a protein or carbohydrate epitope of the target tissue. In one embodiment, the carbohydrate epitope is within a complex carbohydrate, such as one that can bind to a lectin. An exemplary complex carbohydrate is a proteoglycan, including without limitation, chondroitin sulfate, dermatan sulfate, heparin, heparan sulfate, hyaluronate, or keratan sulfate.

In certain embodiments, the targeting moiety comprises a homing peptide. Preferably, the homing peptide selectively directs the progenitor cell to the target tissue. An exemplary homing peptide comprises a sequence selected from PWERSL, FMLRDR, and SGLRQR, and can target to bone marrow tissues. Another exemplary homing peptide comprises a sequence of ASSLNIA, and can target to muscle tissues. Yet another homing peptide comprises a sequence of YSGKWGW, and can target to intestine tissues. Still anther homing peptide comprises a sequence selected from CGFELETC and CGFECVRQCPERC, and can target to lung tissues. In certain embodiments, the targeting moiety comprises an antibody.

Exemplary antibodies include antibodies to type II collagen, chondroitin-4-sulfate, and dermatan sulfate. Optionally, the antibody may be selected from antibodies to collagens I, V, VI and IX, and condroitin-6-sulfate. The antibody may be a monoclonal antibody, a polyclonal antibody, or a humanized antibody.

In certain embodiments, the targeting moiety is a fusion protein. An exemplary fusion protein comprises an Fc fragment. Another exemplary fusion protein comprises a homing peptide. Yet another exemplary fusion protein comprises both an Fc fragment and a homing peptide.

In certain embodiments, the targeting moiety comprises a receptor or a ligand. An exemplary receptor is a chemokine receptor.

In certain embodiments, the targeting moiety comprises an aptamer. In certain embodiments, the targeting moiety is a peptidomimetic.

In certain embodiments, the target tissue is selected from neuronal tissue, connective tissue, hepatic tissue, pancreatic tissue, kidney tissue, bone marrow tissue, cardiac tissue, retinal tissue, intestinal tissue, lung tissue, and endothelium tissue. Optionally, the target tissue is selected from cartilage, skeletal muscle, cardiac muscle, and smooth muscle, bone, tendon, ligament, adipose tissue, and skin.

In certain embodiments, compositions and methods for delivering progenitor cells to target tissues further comprise a bioactive factor. Such bioactive factors can regulate the growth, differentiation, and/or function of the delivered progenitor cell. For example, the bioactive factor may be selected from a transforming growth factor, a bone morphogenetic protein (BMP), a cartilage-derived morphogenic protein, a growth differentiation factor, an angiogenic factor, a platelet-derived growth factor, a vascular endothelial growth factor, an epidermal growth factor, a fibroblast growth factor, a hepatocyte growth factor, an insulin-like growth factor, a nerve growth factor, a colony-stimulating factor (CSF), a neurotrophin (e.g. NT-3, 4 or 5), a growth hormone, an interleukin, a connective tissue growth factor, a parathyroid hormone-related protein, a chemokine, a Wnt protein, a Noggin, and a Gremlin.

In certain embodiments, the progenitor cells having been complexed with a targeting moiety can be delivered to a subject by a variety of methods. For example, the progenitor cell may be delivered to a subject by injection into blood, by injection into the target tissue, by surgical implantation, by subcutaneous injection, by intra-synovial injection, and by intra-peritoneal injection.

Another aspect of the invention provides methods of treating diseases or tissue injuries. Such methods comprise: a) providing a progenitor cell linked to a targeting moiety, wherein the targeting moiety selectively directs the progenitor cell to a diseased or injured target tissue; and b) delivering the progenitor cell linked with the targeting moiety to the diseased or injured target tissue.

In certain embodiments, the progenitor cell is selected from the group consisting of a totipotent stem cell, pluripotent stem cell, multipotent stem cell, mesenchymal stem cell, neuronal stem cell, hematopoietic stem cell, pancreatic stem cell, cardiac stem cell, embryonic stem cell, embryonic germ cell, neural crest stem cell, kidney stem cell, hepatic stem cell, lung stem cell, hemangioblast cell, and endothelial progenitor cell. Optionally, the progenitor cell is selected from a de-differentiated chondrogenic cell, myogenic cell, osteogenic cell, tendogenic cell, ligamentogenic cell, adipogenic cell, and dermatogenic cell.

In certain embodiments, the target tissue of the methods is selected from neuronal tissue, connective tissue, hepatic tissue, pancreatic tissue, kidney tissue, bone marrow tissue, cardiac tissue, retinal tissue, intestinal tissue, lung tissue, and endothelium tissue. Optionally, the target tissue is selected from cartilage, skeletal muscle, cardiac muscle, and smooth muscle, bone, tendon, ligament, adipose tissue, and skin.

In certain embodiments, methods of the invention relate to treating a disease or a tissue injury. For example, the tissue injury may result from laceration, burns, poison or extremes of temperature. Exemplary diseases and injuries may be selected from diabetes, cardiovascular disease, amyotrophic lateral sclerosis, Parkinson's disease, Huntington's disease, multiple sclerosis, stroke, myocardial infarction, spinal cord injury, brain injury, peripheral neuropathy, autoimmune diseases, liver based metabolic diseases, acute liver failure, chronic liver disease, leukemia, sickle-cell anemia, bone defects, muscular dystrophy, burns, osteoarthritis, and macular degeneration.

Other aspects of this invention provide compositions and methods for tissue engineering. In one aspect, the invention provides tissue engineering compositions, which comprise: a) a progenitor cell; b) a targeting moiety that binds to a target tissue; and c) a biocompatible scaffold, wherein the tissue engineering composition generates a scaffold graft to be delivered to a target tissue. In another aspect, the invention provides methods of delivering a scaffold graft in a target tissue. Such methods comprise: a) linking a progenitor cell to a targeting moiety that binds to a target tissue; b) seeding the progenitor cell from (a) onto a biocompatible scaffold, thereby forming a scaffold graft; and c) implanting the scaffold graft from (b) in direct contact with, or adjacent to, a target tissue for a sufficient time, wherein cells of the target tissue associate with the implanted scaffold graft, thereby to form new tissue.

In certain embodiments, the scaffold comprises a bioresorbable material. For example, the bioresorbable material comprises at least one molecule selected from a hydroxy acid, a glycolic acid, caprolactone, hydroxybutyrate, dioxanone, an orthoester, an orthocarbonate, or an aminocarbonate, collagen, cellulose, fibrin, hyaluronic acid, fibronectin, chitosan.

In other embodiments, the scaffold comprises a non-bioresorbable material. For example, the non-bioresorbable material comprises at least one molecule selected from a polyalkylene terephthalate, a polyamide, a polyalkene, poly(vinyl fluoride), polytetrafluoroethylene carbon fibers, natural or synthetic silk, carbon fiber, and glass.

In certain embodiments, compositions and methods for tissue engineering further comprise a bioactive factor. For example, the bioactive factor is selected from a transforming growth factor, a bone morphogenetic protein (BMP), a cartilage-derived morphogenic protein, a growth differentiation factor, an angiogenic factor, a platelet-derived growth factor, a vascular endothelial growth factor, an epidermal growth factor, a fibroblast growth factor, a hepatocyte growth factor, an insulin-like growth factor, a nerve growth factor, a colony-stimulating factor (CSF), a neurotrophin (e.g., NT-3, 4 or 5), a growth hormone, an interleukin, a connective tissue growth factor, a parathyroid hormone-related protein, a chemokine, a Wnt protein, a Noggin, and a Gremlin. Such bioactive factors can regulate the growth, differentiation, and/or function of the progenitor cell employed in tissue engineering.

In certain embodiments, the progenitor cell is selected from the group consisting of a totipotent stem cell, pluripotent stem cell, multipotent stem cell, mesenchymal stem cell, neuronal stem cell, hematopoietic stem cell, pancreatic stem cell, cardiac stem cell, embryonic stem cell, embryonic germ cell, neural crest stem cell, kidney stem cell, hepatic stem cell, lung stem cell, hemangioblast cell, and endothelial progenitor cell. Optionally, the progenitor cell is selected from a de-differentiated chondrogenic cell, myogenic cell, osteogenic cell, tendogenic cell, ligamentogenic cell, adipogenic cell, and dermatogenic cell.

In certain embodiments, the progenitor cell is pre-coated with a linker before linked to a targeting moiety. An exemplary linker includes a protein G or a protein A. Optionally, the linker is modified with a lipophilic moiety which includes without limitation, a palmitoyl moiety, myristoyl moiety, margaroyl moiety, stearoyl moiety, arachidoyl moiety, acetyl moiety, butytyl moiety, hexanoyl moiety, octanoyl moiety, decanoyl moiety, lauroyl moiety, palmitoleoyl moiety, behenoyl moiety, and lignoceroyl moiety. A preferred lipophilic moiety is a palmitoyl moiety, a myristoyl moiety or a margaroyl moiety.

In other embodiments, the progenitor cell is directly linked to a targeting moiety. Optionally, the targeting moiety is modified with a lipophilic moiety which includes without limitation, a palmitoyl moiety, a myristoyl moiety, a margaroyl moiety, a stearoyl moiety, an arachidoyl moiety, an acetyl moiety, a butytyl moiety, a hexanoyl moiety, an octanoyl moiety, a decanoyl moiety, a lauroyl moiety, a palmitoleoyl moiety, a behenoyl moiety, and a lignoceroyl moiety. A preferred lipophilic moiety is a palmitoyl moiety, a myristoyl moiety or a margaroyl moiety.

In certain embodiments, the progenitor cell expresses a cell surface marker or an extracellular matrix molecule, for example, CD4, CD8, CD10, CD30, CD33, CD34, CD38, CD45, CD133, CD146, fetal liver kinase-1 (Flk1), C-Kit, Lin, Mac-1, Sca-1, Stro-1, Thy-1, Collagen types II or IV, O1, O4, N-CAM, p75, or SSEA.

In certain embodiments, the targeting moiety comprises a component of a specific binding pair. Preferably, the targeting moiety interacts with an epitope intrinsic to the target tissue. Optionally, the epitope may be a protein or carbohydrate epitope of the target tissue. In one embodiment, the carbohydrate epitope is within a complex carbohydrate, such as one that can bind to a lectin. An exemplary complex carbohydrate is a proteoglycan, including without limitation, chondroitin sulfate, dermatan sulfate, heparin, heparan sulfate, hyaluronate, or keratan sulfate.

In certain embodiments, the targeting moiety comprises a homing peptide. Preferably, the homing peptide selectively directs the progenitor cell to the target tissue. An exemplary homing peptide comprises a sequence selected from PWERSL, FMLRDR, and SGLRQR, and can target to bone marrow tissues. Another exemplary homing peptide comprises a sequence of ASSLNIA, and can target to muscle tissues. Yet another homing peptide comprises a sequence of YSGKWGW, and can target to intestine tissues. Still anther homing peptide comprises a sequence selected from CGFELETC and CGFECVRQCPERC, and can target to lung tissues.

In certain embodiments, the targeting moiety comprises an antibody. Exemplary antibodies include antibodies to type II collagen, chondroitin-4-sulfate, and dermatan sulfate. Optionally, the antibody may be selected from antibodies to collagens I, V, VI and IX, and condroitin-6-sulfate. The antibody may be a monoclonal antibody, a polyclonal antibody or a humanized antibody.

In certain embodiments, the targeting moiety is a fusion protein. An exemplary fusion protein comprises an Fc fragment. Another exemplary fusion protein comprises a homing peptide. Yet another exemplary fusion protein comprises both an Fc fragment and a homing peptide.

In certain embodiments, the targeting moiety comprises a receptor or a ligand. An exemplary receptor is a chemokine receptor.

In certain embodiments, the targeting moiety comprises an aptamer. In certain embodiments, the targeting moiety is a peptidomimetic.

In certain embodiments, the target tissue is selected from neuronal tissue, connective tissue, hepatic tissue, pancreatic tissue, kidney tissue, bone marrow tissue, cardiac tissue, retinal tissue, intestinal tissue, lung tissue, and endothelium tissue. Optionally, the target tissue is selected from cartilage, skeletal muscle, cardiac muscle, and smooth muscle, bone, tendon, ligament, adipose tissue, and skin.

In certain embodiments, the scaffold graft can be delivered to the target tissue by a variety of methods, for example, by surgical implantation. In other embodiments, such methods may further comprise removing the scaffold graft from the subject.

The embodiments and practices of the present invention, other embodiments, and their features and characteristics, will be apparent from the description, figures and claims that follow, with all of the claims hereby being incorporated by this reference into this Summary.

### BRIEF DESCRIPTION OF THE DRAWINGS

The file of this patent contains at least one drawing executed in color. Copies of this patent with color drawing(s) will be provided by the Patent and Trademark Office upon request and payment of the necessary fee.

FIG. 1. The graph shows the concentration of palmitated protein G (PPG) (or lipidated antibody binding protein, LAB-P) on the x-axis, and the relative fluorescence intensity in arbitrary units on the y-axis. The insert shows fluorescent micrographs of representative cells coated with PPG.

FIG. 2. Cell viability is indicated as a percentage of the starting cell number prior to treating with LAB-P (PPG). No significant loss of cell numbers were observed at any concentration of LAB-P that was used.

FIG. 3. Cells coated with different concentrations of LAB-P were cultured for one week in standard culture medium and were shown to expand approximately 3 fold at all coating concentrations tested.

FIG. 4. Pre-chondrocytes coated with 60 μg/ml of PPG were cultured in chondrogenic conditions for 3 weeks, harvested, fixed, embedded, sectioned and then stained for collagen type II. The intense purple staining indicates the presence of type II collagen indicating that the coating procedure has not interfered with the ability of these cells to differentiate into chondrocytes. The sample was counter-stained with Fast Green.

FIG. 5. Targeting of Vybrant™-stained cells to frozen sections of cartilage. Vybrant™ cells (green) are shown on the surface of the rabbit articular cartilage sections; cartilage nuclei are stained red with propidium iodide. Upper left shows control cells (PPG only); only a few cells adhere. Upper right shows cells coated with PPG+anti-type II collagen antibody. Lower left shows PPG+anti-chondroitin-4-sulfate. Lower right shows PPG+anti-keratan sulfate. Each of the three samples incubated with cells containing the targeting antibody had more Vybrant™-positive cells than did control.

FIG. 6. Explants of rabbit articular cartilage incubated with Vybrant™-stained cells. Upper left shows cells' coated with PPG only. Only one cell is visible on the left side of the u-shaped defect. Upper right is chondoroitin-1-sulfate antibody. Lower left is collagen II (arrow points to insert showing a higher magnification of Vybrant™-positive cells). Lower right is both antibodies in combination. Each of the antibody coated cell preparations showed greater numbers of positive cells in the PPG only control.

FIG. 7. Confocal imaging of targeted cartilage explants. Fluorescently-labeled cells (incubated in 10 μM Vybrant™) with and without pre-coating with antibodies were incubated with cartilage explants, washed in buffer, fixed and then observed and analyzed by confocal microscopy. Optical slices were collected at 30 μm intervals from the bottom of the defect to the articular surface and the images were processed by Z-stacking using Zeiss LSM software. Control cells coated with PPG only (A) showed nearly no affinity for undamaged cartilage surfaces while the defect area had a low fluorescent signal. Cells coated with an antibody to type II collagen (B) generally showed a moderate increase in fluorescent signal compared to PPG only. Cells coated with two antibodies (C) such as anti-type II collagen and anti-chodroitin-4-sulfate (2B6) showed a much greater intensity of fluorescence than PPG only samples, as did coating with triple antibodies (D) to type II collagen, chondroitin-4-sulfate and keratan sulfate (5D4).

FIG. 8. Quantification of confocal microscopic fluorescent signal in rabbit cartilage explants. Fluorescent microscopic images were collected throughout the depth of the cartilage defect and Z-stack images formed with Zeiss LSM software. The intensity of the Z-stacked images was analyzed with Metamorph software and the intensities with the defect area (dark circles) and the cartilage surface (open circles) was determined for 4 separate samples. The fluorescent signal within the defect was always greater than that detected on the undamaged cartilage surface in all samples. There was a trend of increasing fluorescent signal in single antibody coated samples, but only samples coated with antibodies to both chondroitin-4-sulfate (2B6) and collagen II (TIIC) or triple-coated with 2B6, TIIC and antibody to keratan sulfate (5D4) showed a significant increase in intensity compared to controls at p<0.05 (ANOVA).

### DETAILED DESCRIPTION OF THE INVENTION

1. Definitions

For convenience, certain terms employed in the specification, examples, and appended claims are collected here. Unless defined otherwise, all technical and scientific terms used herein have the same meaning as commonly understood by one of ordinary skill in the art to which this invention belongs.

The articles “a” and “an” are used herein to refer to one or to more than one (i.e., to at least one) of the grammatical object of the article. By way of example, “an element” means one element or more than one element.

The term “antibody” refers to an immunoglobulin, derivatives thereof which maintain specific binding ability, and proteins having a binding domain which is homologous or largely homologous to an immunoglobulin binding domain. These proteins may be derived from natural sources, or partly or wholly synthetically produced. An antibody may be monoclonal or polyclonal. The antibody may be a member of any immunoglobulin class, including any of the human classes: IgG, IgM, IgA, IgD, and IgE. In exemplary embodiments, antibodies used with the methods and compositions described herein are derivatives of the IgG class.

The term “antibody fragment” refers to any derivative of an antibody which is less than full-length. In exemplary embodiments, the antibody fragment retains at least a significant portion of the full-length antibody's specific binding ability. Examples of antibody fragments include, but are not limited to, Fab, Fab′, F(ab′)₂, scFv, Fv, dsFv diabody, and Fd fragments. The antibody fragment may be produced by any means. For instance, the antibody fragment may be enzymatically or chemically produced by fragmentation of an intact antibody, it may be recombinantly produced from a gene encoding the partial antibody sequence, or it may be wholly or partially synthetically produced. The antibody fragment may optionally be a single chain antibody fragment. Alternatively, the fragment may comprise multiple chains which are linked together, for instance, by disulfide linkages. The fragment may comprise chains synthesized from engineered DNA sequences that have been modified by, for instance, substituting one amino acid for another to eliminate disulfide linkage sites. The fragment may also optionally be a multimolecular complex. A functional antibody fragment will typically comprise at least about 50 amino acids and more typically will comprise at least about 200 amino acids.

The term “chondrogenic cells” includes chondrocytes and cells that differentiate into chondrocytes. The term may also refer to cells that are genetically altered or otherwise manipulated so as to become cells that produce substantial components of the cartilage matrix.

The term “complex carbohydrates” herein include proteoglycans such as chondroitin sulfate, dermatan sulfate, heparin, heparan sulfate, hyaluronate, and keratan sulfate. The complex carbohydrates also include those polysaccharides which can be bound by lectins.

The term “diabodies” refers to dimeric scFvs. The components of diabodies typically have shorter peptide linkers than most scFvs and they show a preference for associating as dimers.

As used herein, the term “epitope” refers to a physical structure on a molecule that interacts with a selective component. In exemplary embodiments, epitope refers to a desired region on a target molecule that specifically interacts with a selectivity component.

The term “Fab” refers to an antibody fragment that is essentially equivalent to that obtained by digestion of immunoglobulin (typically IgG) with the enzyme papain. The heavy chain segment of the Fab fragment is the Fd piece. Such fragments may be enzymatically or chemically produced by fragmentation of an intact antibody, recombinantly produced from a gene encoding the partial antibody sequence, or it may be wholly or partially synthetically produced.

The term “Fab′” refers to an antibody fragment that is essentially equivalent to that obtained by reduction of the disulfide bridge or bridges joining the two heavy chain pieces in the F(ab′)₂ fragment. Such fragments may be enzymatically or chemically produced by fragmentation of an intact antibody, recombinantly produced from a gene encoding the partial antibody sequence, or it may be wholly or partially synthetically produced.

The term “F(ab′)₂” refers to an antibody fragment that is essentially equivalent to a fragment obtained by digestion of an immunoglobulin (typically IgG) with the enzyme pepsin at pH 4.04.5. Such fragments may be enzymatically or chemically produced by fragmentation of an intact antibody, recombinantly produced from a gene encoding the partial antibody sequence, or it may be wholly or partially synthetically produced.

The term “Fv” refers to an antibody fragment that consists of one V_{H} and one V_{L} domain held together by noncovalent interactions. The term “dsFv” is used herein to refer to an Fv with an engineered intermolecular disulfide bond to stabilize the V_{H}-V_{L} pair.

As used herein, the term “homing peptide” refers to a particular peptide that binds relatively specifically to an epitope of a target tissue or organ, following administration to a subject In general, a homing peptide that selectively homes to a target tissue is characterized, in part, by detecting at least a 2-fold greater specific binding of the peptide to the target tissue as compared to a control tissue.

The term “immunogen” traditionally refers to compounds that are used to elicit an immune response in an animal, and is used as such herein. However, many techniques used to produce a desired selectivity component, such as the phage display and aptamer methods described below, do not rely wholly, or even in part, on animal immunizations. Nevertheless, these methods use compounds containing an “epitope,” as defined above, to select for and clonally expand a population of selectivity components specific to the “epitope.” These in vitro methods mimic the selection and clonal expansion of immune cells in vivo, and, therefore, the compounds containing the “epitope” that is used to clonally expand a desired population of phage, aptamers and the like in vitro are embraced within the definition of “immunogens.”

As used herein, the term “lipophilic moiety” includes any lipid soluble long-chain fatty acid. For example, the lipophilic moiety includes a palmitoyl moiety, a myristoyl moiety, a margaroyl moiety, a stearoyl moiety, an arachidoyl moiety, an acetyl moiety, a butytyl moiety, a hexanoyl moiety, an octanoyl moiety, a decanoyl moiety, a lauroyl moiety, a palmitoleoyl moiety, a behenoyl moiety, and a lignoceroyl moiety.

The term “progenitor cell” as used herein, includes any totipotent stem cell, pluripotent stem cell, and multipotent stem cell, as well as any of their lineage descendant cells. The terms “stem cell” and “progenitor cell” are used interchangeably herein. The progenitor cell can derive from either embryonic tissues or adult tissues. Exemplary progenitor cells can be selected from, but not restricted to, totipotent stem cell, pluripotent stem cell, multipotent stem cell, mesenchymal stem cell, neuronal stem cell, hematopoietic stem cell, pancreatic stem cell, cardiac stem cell, embryonic stem cell, embryonic germ cell, neural crest stem cell, kidney stem cell, hepatic stem cell, lung stem cell, hemangioblast cell, and endothelial progenitor cell. Additional exemplary progenitor cells are selected from, but not restricted to, de-differentiated chondrogenic cell, myogenic cell, osteogenic cell, tendogenic cell, ligamentogenic cell, adipogenic cell, and dermatogenic cell.

The terms “single-chain Fvs” and “scFvs” refers to recombinant antibody fragments consisting of only the variable light chain (V_{L}) and variable heavy chain (V_{H}) covalently connected to one another by a polypeptide linker. Either V_{L} or V_{H} may be the NH₂-terminal domain. The polypeptide linker may be of variable length and composition so long as the two variable domains are bridged without serious steric interference. In exemplary embodiments, the linkers are comprised primarily of stretches of glycine and serine residues with some glutamic acid or lysine residues interspersed for solubility.

As used herein, the term “targeting moiety” refers to a moiety capable of interacting with a target molecule. Targeting moieties having limited cross-reactivity are generally preferred. In certain embodiments, suitable targeting moieties include, for example, any member of a specific binding pair, antibodies, monoclonal antibodies, or derivatives or analogs thereof, including without limitation: Fv fragments, single chain Fv (scFv) fragments, Fab′ fragments, F(ab′)2 fragments, single domain antibodies, camelized antibodies and antibody fragments, humanized antibodies and antibody fragments, and multivalent versions of the foregoing; multivalent binding reagents including without limitation: monospecific or bispecific antibodies, such as disulfide stabilized Fv fragments, scFv tandems ((scFv)₂ fragments), diabodies, tribodies or tetrabodies, which typically are covalently linked or otherwise stabilized (i.e., leucine zipper or helix stabilized) scFv fragments; and other targeting moieties include for example, homing peptides, fusion proteins, receptors, ligands, aptamers, and peptidomimetics.

2. Overview

In certain embodiments, the approach described herein is based on a cell coating technique that generates delivery compositions comprising a cell and a targeting moiety, where the targeting moiety is designed to bind to a target location, such as a tissue, extracellular matrix, cell type, etc. In certain embodiments, the cell is a chondrogenic cell and the targeting moiety binds cartilage matrix. The cell coating technique enhances adherence of chondrogenic cells, such as osteochondral progenitor cells, to cartilage matrix injury site by coating the cells with matrix specific antibodies. We show that this enhanced adherence of cells increases the number of chondrogenic cells at the articular injury site, and, while not wishing to be bound to theory, it is expected that, the increased presence of cells at the injury site shifts the balance of the repair process into a net anabolic one.

In certain embodiments, the cell coating technique employs a linker to connect the cell to the targeting moiety. For example, protein A and protein G are useful linkers where the targeting moiety comprises an antibody. In certain embodiments, the linker is connected to the cell by a lipophilic moiety, as in the case of palmitated protein A or protein G. The lipophilic moiety of the palmitate hydrocarbon chains makes it possible to coat the cell membrane with this linker by insertion into the outer leaflet of the phospholipid bilayer.

3. Progenitor Cells

In certain aspects, the present invention provides compositions and methods comprising a progenitor cell. As described herein, any progenitor cell that is suitable for the targeted tissue, matrix, etc. may be employed, including any totipotent stem cell, pluripotent stem cell, and multipotent stem cell, as well as any of their lineage descendant cells. The progenitor cell may derive from either embryonic tissues or adult tissues. In certain embodiments, the progenitor cell is selected from totipotent stem cell, pluripotent stem cell, multipotent stem cell, mesenchymal stem cell, neuronal stem cell, hematopoietic stem cell, pancreatic stem cell, cardiac stem cell, embryonic stem cell, embryonic germ cell, neural crest stem cell, kidney stem cell, hepatic stem cell, lung stem cell, hemangioblast cell, and endothelial progenitor cell. In other embodiments, the progenitor cell is selected from de-differentiated chondrogenic cell, myogenic cell, osteogenic cell, tendogenic cell, ligamentogenic cell, adipogenic cell, and dermatogenic cell.

Exemplary progenitor cells and methods for obtaining such cells are well known in the art and described in the following U.S. patents (prefaced by “US”) and international patent applications (prefaced by “WO”): U.S. Pat. No. 5,130,141; U.S. Pat. No. 5,453,357; U.S. Pat. No. 5,486,359; U.S. Pat. No. 5,589,376; U.S. Pat. No. 5,723,331; U.S. Pat. No. 5,736,396; U.S. Pat. No. 5,843,780; U.S. Pat. No. 5,877,299; U.S. Pat. No. 5,827,735; U.S. Pat. No. 5,906,934; U.S. Pat. No. 5,980,887; U.S. Pat. No. 6,200,806; U.S. Pat. No. 6,214,369; U.S. Pat. No. 6,429,012; WO 00/53795; WO 00/02654; WO 00/78929; WO 01/11011; WO 01/42425; WO 02/86082.

In certain preferred embodiments, the progenitor cell is a chondrogenic cell. Exemplary chondrogenic cells include chondrocytes such as articular chondrocytes. In certain embodiments, chondrocytes may be identified by toluidine blue staining, where chondrocytes are surrounded by meta-chromatic staining representing highly sulfated glycosaminoglycans. Chondrogenic cells also include cells that can differentiate or give rise to chondrocytes. Exemplary cells that differentiate to form chondrocytes or give rise to chondrocytes include mesenchymal stem cells, stem cells derived from adipose tissue, osteochondral progenitor cells; embryonic stem cells; multipotent adult stem cells, etc.

In certain embodiments, the progenitor cell expresses a cell surface marker or an extracellular matrix molecule. For example, the endothelial progenitor cell expresses a cell surface marker, i.e., fetal liver kinase-1 (Flk1). Another exemplary cell surface marker is p75 (a low affinity nerve growth factor receptor) for the neural crest stem cell. The cell surface marker or extracellular matrix molecule can be selected from, but not limited to, CD4, CD8, CD10, CD30, CD33, CD34, CD38, CD45, CD133, CD146, fetal liver kinase-1 (Flk1), C-Kit, Lin, Mac-1, Sca-1, Stro-1, Thy-1, Collagen types II or IV, O1, O4, N-CAM, p75, and SSEA.

In certain embodiments, the progenitor cells are immunologically matched to the subject who will receive them (e.g., similar HLA typing), and optionally, the cells are autologous, meaning that they are derived from the subject.

In certain embodiments, progenitor cells may be harvested and stored (e.g., by cryogen freezing), allowing banking of cells for later use.

4. Target Tissues

In certain aspects, the present invention provides compositions and methods comprising a target tissue. As one skilled in the art would appreciate, any target tissue that is suitable for a progenitor cell delivery may be employed, wherein the delivered progenitor cell is capable of self-renewing and regenerates the target tissue. In certain embodiments, the target tissue can be selected from neuronal tissue (including both neuron and glia), connective tissue, hepatic tissue, pancreatic tissue, kidney tissue, bone marrow tissue, cardiac tissue, retinal tissue, intestinal tissue, lung tissue, and endothelium tissue. In other embodiments, the target tissue can be selected from cartilage, skeletal muscle, cardiac muscle, smooth muscle, bone, tendon, ligament, adipose tissue, and skin. Preferably, the target tissue may be entirely or partially damaged by a disease or an injury.

5. Targeting Moieties

In certain aspects, the present invention provides compositions and methods comprising a targeting moiety. The targeting moiety may be any molecule, or complex of molecules, which is capable of interacting with a desired target, including, for example, a tissue, a cell type, an extracellular matrix, a carbohydrates, a protein, etc. Exemplary targeting moieties may include, for example, antibodies, antibody fragments, homing peptides, non-antibody receptors, ligands, aptamers, peptidomimetics, etc. A targeting moiety may include additional components that assist in forming an attachment between the targeting moiety and a coated cell. Targeting moieties having limited cross-reactivity are generally preferred.

In certain embodiments, the targeting moiety used to deliver a progenitor cell to a target tissue interact with an epitope intrinsic to the target tissue. Such epitopes can be either protein epitopes or carbohydrate epitopes of the target tissues. For example, when the target tissue is cartilage, the epitope for a targeting moiety can be any available antigen selected from the primary extracellular matrix molecules contained in cartilage. A primary epitope for promoting chondrocyte cell attachment is type II collagen, which is the most abundant fibrillar collagen in cartilage. The next most prominent molecules, based on dry weight, are the proteoglycans, which represent 20-30% of the cartilage dry weight. Although abundant, collagen type II fibers are masked by other molecules, especially proteoglycan molecules that are often observed to be in direct contact with the collagen fibers. As a percentage of volume proteoglycans are much more abundant than collagen type II and in addition, it is known from structural and biochemical analysis of proteoglycans that there are hundreds of chondroitin sulfate and keratan sulfate side chains on each aggrecan molecule, and since each glycosaminoglycan side chain can have multiple antigenic epitopes, proteoglycans are key targets for these cell-binding strategies.

(a) Antibodies

In certain embodiments, a targeting moiety of the invention may comprise an antibody, including a monoclonal antibody, a polyclonal antibody, and a humanized antibody. Such antibody can bind to an antigen of a target tissue and thus mediate the delivery of a progenitor cell to the target tissue. For example, antibodies can be selected that are most likely to bind to cartilage matrix based on a previous study from this laboratory indicating the accessibility of chondroitin and keratan-sulfate chains to antibody binding before and after moderate chondroitinase ABC digestion. Preferred antibodies include antibodies to type II collagen, chondroitin-4-sulfate, or dermatan sulfate. Other preferred antibodies include antibodies to collagens I, V, VI or IX, and antibodies to condroitin-6-sulfate, or a combination of the different antibodies.

In some embodiments, targeting moieties may comprise antibody fragments, derivatives or analogs thereof, including without limitation: Fv fragments, single chain Fv (scFv) fragments, Fab′ fragments, F(ab′)2 fragments, single domain antibodies, camelized antibodies and antibody fragments, humanized antibodies and antibody fragments, and multivalent versions of the foregoing; multivalent targeting moieties including without limitation: monospecific or bispecific antibodies, such as disulfide stabilized Fv fragments, scFv tandems ((scFv)₂ fragments), diabodies, tribodies or tetrabodies, which typically are covalently linked or otherwise stabilized (i.e., leucine zipper or helix stabilized) scFv fragments; receptor molecules which naturally interact with a desired target molecule.

Preparation of antibodies may be accomplished by any number of well-known methods for generating monoclonal antibodies. These methods typically include the step of immunization of animals, typically mice, with a desired immunogen (e.g., a desired target molecule or fragment thereof). Once the mice have been immunized, and preferably boosted one or more times with the desired immunogen(s), monoclonal antibody-producing hybridomas may be prepared and screened according to well known methods (see, for example, Kuby, Janis, Immunology, Third Edition, pp. 131-139, W.H. Freeman & Co. (1997), for a general overview of monoclonal antibody production, that portion of which is incorporated herein by reference).

Over the past several decades, antibody production has become extremely robust. In vitro methods that combine antibody recognition and phage display techniques allow one to amplify and select antibodies with very specific binding capabilities. See, for example, Holt, L. J. et al., “The Use of Recombinant Antibodies in Proteomics,” Current Opinion in Biotechnology, 2000, 11:445-449, incorporated herein by reference. These methods typically are much less cumbersome than preparation of hybridomas by traditional monoclonal antibody preparation methods. Binding epitopes may range in size from small organic compounds such as bromo uridine and phosphotyrosine to oligopeptides on the order of 7-9 amino acids in length.

In one embodiment, phage display technology may be used to generate a targeting moiety specific for a desired target molecule. An immune response to a selected immunogen is elicited in an animal (such as a mouse, rabbit, goat or other animal) and the response is boosted to expand the immunogen-specific B-cell population. Messenger RNA is isolated from those B-cells, or optionally a monoclonal or polyclonal hybridoma population. The mRNA is reverse-transcribed by known methods using either a poly-A primer or murine immunoglobulin-specific primer(s), typically specific to sequences adjacent to the desired V_{H} and V_{L} chains, to yield cDNA. The desired V_{H} and V_{L} chains are amplified by polymerase chain reaction (PCR) typically using V_{H} and V_{L} specific primer sets, and are ligated together, separated by a linker. V_{H} and V_{L} specific primer sets are commercially available, for instance from Stratagene, Inc. of La Jolla, Calif. Assembled V_{H}-linker-V_{L} product (encoding an scFv fragment) is selected for and amplified by PCR. Restriction sites are introduced into the ends of the V_{H}-linker-V_{L} product by PCR with primers including restriction sites and the scFv fragment is inserted into a suitable expression vector (typically a plasmid) for phage display. Other fragments, such as an Fab′ fragment, may be cloned into phage display vectors for surface expression on phage particles. The phage may be any phage, such as lambda, but typically is a filamentous phage, such as fd and M13, typically M13.

In phage display vectors, the V_{H}-linker-V_{L} sequence is cloned into a phage surface protein (for M13, the surface proteins g3p (pIII) or g8p, most typically g3p). Phage display systems also include phagemid systems, which are based on a phagemid plasmid vector containing the phage surface protein genes (for example, g3p and g8p of M13) and the phage origin of replication. To produce phage particles, cells containing the phagemid are rescued with helper phage providing the remaining proteins needed for the generation of phage. Only the phagemid vector is packaged in the resulting phage particles because replication of the phagemid is grossly favored over replication of the helper phage DNA. Phagemid packaging systems for production of antibodies are commercially available. One example of a commercially available phagemid packaging system that also permits production of soluble ScFv fragments in bacteria cells is the Recombinant Phage Antibody System (RPAS), commercially available from Amersham Pharmacia Biotech, Inc. of Piscataway, N.J. and the pSKAN Phagemid Display System, commercially available from MoBiTec, LLC of Marco Island, Fla. Phage display systems, their construction and screening methods are described in detail in, among others, U.S. Pat. Nos. 5,702,892, 5,750,373, 5,821,047 and 6,127,132, each of which are incorporated herein by reference in their entirety.

A targeting moiety need not originate from a biological source. A targeting moiety may, for example, be screened from a combinatorial library of synthetic peptides. One such method is described in U.S. Pat. No. 5,948,635, incorporated herein by reference, which described the production of phagemid libraries having random amino acid insertions in the pIII gene of M13. These phage may be clonally amplified by affinity selection as described above.

The immunogens used to prepare targeting moieties having a desired specificity will generally be the target molecule, or a fragment or derivative thereof. Such immunogens may be isolated from a source where they are naturally occurring or may be synthesized using methods known in the art. For example, peptide chains may be synthesized by 1-ethyl-3-[dimethylaminopropyl] carbodiimide (EDC)-catalyzed condensation of amine and carboxyl groups. It certain embodiments, the immunogen may be linked to a carrier bead or protein. For example, the carrier may be a functionalized bead such as SASRIN™ resin commercially available from Bachem, King of Prussia, Pa. or a protein such as keyhole limpet hemocyanin (KLH) or bovine serum albumin (BSA). The immunogen may be attached directly to the carrier or may be associated with the carrier via a linker, such as a non-immunogenic synthetic linker (for example, a polyethylene glycol (PEG) residue, amino caproic acid or derivatives thereof) or a random, or semi-random polypeptide.

In certain embodiments, it may be desirable to mutate the binding region of a polypeptide targeting moiety and select for a targeting moiety with superior binding characteristics as compared to the un-mutated targeting moiety. This may be accomplished by any standard mutagenesis technique, such as by PCR with Taq polymerase under conditions that cause errors. In such a case, the PCR primers could be used to amplify scFv-encoding sequences of phagemid plasmids under conditions that would cause mutations. The PCR product may then be cloned into a phagemid vector and screened for the desired specificity, as described above.

In other embodiments, the targeting moieties may be modified to make them more resistant to cleavage by proteases. For example, the stability of targeting moiety comprising a polypeptide may be increased by substituting one or more of the naturally occurring amino acids in the (L) configuration with D-amino acids. In various embodiments, at least 1%, 5%, 10%, 20%, 50%, 80%, 90% or 100% of the amino acid residues of targeting moiety may be of the D configuration. The switch from L to D amino acids neutralizes the digestion capabilities of many of the ubiquitous peptidases found in the digestive tract. Alternatively, enhanced stability of a targeting moiety comprising a peptide bond may be achieved by the introduction of modifications of the traditional peptide linkages. For example, the introduction of a cyclic ring within the polypeptide backbone may confer enhanced stability in order to circumvent the effect of many proteolytic enzymes known to digest polypeptides in the stomach or other digestive organs and in serum. In still other embodiments, enhanced stability of a targeting moiety may be achieved by intercalating one or more dextrorotatory amino acids (such as, dextrorotatory phenylalanine or dextrorotatory tryptophan) between the amino acids of targeting moiety. In exemplary embodiments, such modifications increase the protease resistance of a targeting moiety without affecting the activity or specificity of the interaction with a desired target molecule.

In certain embodiments, the antibodies or variants thereof, may be modified to make them less immunogenic when administered to a subject. For example, if the subject is human, the antibody may be “humanized”; where the complimentarily determining region(s) of the hybridoma-derived antibody has been transplanted into a human monoclonal antibody, for example as described in Jones, P. et al. (1986), Nature, 321, 522-525 or Tempest et al. (1991), Biotechnology, 9, 266-273. Also, transgenic mice, or other mammals, may be used to express humanized antibodies. Such humanization may be partial or complete.

(b) Homing Peptides

In certain embodiments, a targeting moiety of the present invention may comprise a homing peptide which selectively direct a progenitor cell to a target tissue. For example, delivering a progenitor cell to the lung can be mediated by a homing peptide comprising an amino acid sequence of CGFELETC or CGFECVRQCPERC. Further exemplary homing peptide sequences and their target tissues are listed in Table I.

TABLE I
Exemplary homing peptide sequences and their target tissues.

| Targeted Tissues | Homing Peptide Sequences |
|---|---|
| Bone Marrow | PWERSL |
| | FMLRDR |
| | SGLRQR |
| Lung | CGFELETC |
| | CGFECVRQCPERC |
| Muscle | ASSLNIA |
| Intestine | YSGKWGW |

Homing peptides for a target tissue (or organ) can be identified using various methods well known in the art. An exemplary method is the in vivo phage display method. Specifically, random peptide sequences are expressed as fusion peptides with the surface proteins of phage, and this library of random peptides are infused into the systemic circulation. After infusion into host mice, target tissues or organs are harvested, the phage is then isolated and expanded, and the injection procedure repeated two more times. Each round of injection includes, by default, a negative selection component, as the injected virus has the opportunity to either randomly bind to tissues, or to specifically bind to non-target tissues. Virus sequences that specifically bind to non-target tissues will be quickly eliminated by the selection process, while the number of non-specific binding phage diminishes with each round of selection. Many laboratories have identified the homing peptides that are selective for vasculature of brain, kidney, lung, skin, pancreas, intestine, uterus, adrenal gland, retina, muscle, prostate, or tumors. See, for example, Samoylova et al., 1999, Muscle Nerve, 22:460; Pasqualini et al., 1996, Nature, 380:364; Koivunen et al., 1995, Biotechnology, 13:265; Pasqualini et al., 1995, J. Cell Biol., 130:1189; Pasqualini et al., 1996, Mole. Psych., 1:421, 423; Rajotte et al., 1998, J. Clin. Invest., 102:430; Rajotte et al., 1999, J. Biol. Chem., 274:11593. See, also, U.S. Pat. Nos. 5,622,699; 6,068,829; 6,174,687; 6,180,084; 6,232,287; 6,296,832; 6,303,573; 6,306,365.

Phage display technology provides a means for expressing a diverse population of random or selectively randomized peptides. Various methods of phage display and methods for producing diverse populations of peptides are well known in the art. For example, methods for preparing diverse populations of binding domains on the surface of a phage have been described in U.S. Pat. No. 5,223,409. In particular, phage vectors useful for producing a phage display library as well as methods for selecting potential binding domains and producing randomly or selectively mutated binding domains are also provided in U.S. Pat. No. 5,223,409. Similarly, methods of producing phage peptide display libraries, including vectors and methods of diversifying the population of peptides that are expressed, are also described in Smith et al., 1993, Meth. Enzymol., 217:228-257, Scott et al., Science, 249:386-390, and two PCT publications WO 91/07141 and WO 91/07149. Phage display technology can be particularly powerful when used, for example, with a codon based mutagenesis method, which can be used to produce random peptides or randomly or desirably biased peptides (see, e.g., U.S. Pat. No. 5,264,563). These or other well-known methods can be used to produce a phage display library, which can be subjected to the in vivo phage display method in order to identify a peptide that homes to one or a few selected tissues.

In vitro screening of phage libraries has previously been used to identify peptides that bind to antibodies or to cell surface receptors (see, e.g., Smith et al., 1993, Meth. Enzymol., 217:228-257). For example, in vitro screening of phage peptide display libraries has been used to identify novel peptides that specifically bind to integrin adhesion receptors (see, e.g., Koivunen et al., 1994, J. Cell Biol 124:373-380), and to the human urokinase receptor (Goodson et al., 1994, Proc. Natl. Acad. Sci., USA 91:7129-7133). However, such in vitro studies provide no insight as to whether a peptide that can specifically bind to a selected receptor in vitro also will bind the receptor in vivo or whether the binding peptide or the receptor are unique to a specific organ in the body.

(c) Fusion Proteins

In certain embodiments, a targeting moiety of the invention may be a fusion protein. Such fusion protein may contain a tag that facilitates its isolation, immobilization, identification, or detection and/or which increases its solubility. In a preferred embodiment, the fusion protein comprises an Fc fragment of antibodies. The Fc fragment can bind to a Protein A or Protein G. In another preferred embodiment, the fusion protein comprises a homing peptide which selectively directs a progenitor cell to a target tissue. An exemplary fusion protein comprises a homing peptide fused to the amino terminus of the Fc region of the human IgG sequence and to the carboxyl terminus of the oncostatin-M signal peptide.

The fusion protein may contain other tags, for example, glutathione S-transferase (GST), calmodulin-binding peptide, thioredoxin, maltose binding protein, HA, myc, poly arginine, poly His, poly His-Asp or FLAG tags. Additional exemplary tags include polypeptides that alter protein localization in vivo, such as signal peptides, type III secretion system-targeting peptides, transcytosis domains, nuclear localization signals, etc. In various embodiments, a targeting moiety of the invention may comprise one or more tags, including multiple copies of the same tag or two or more different tags. It is also within the scope of the invention to include a spacer (such as a polypeptide sequence or a chemical moiety) between a targeting moiety of the invention and the tag in order to facilitate construction or to optimize its structural constraints. In another embodiment, the tagged moiety may be constructed so as to contain protease cleavage sites between the tag and the moiety in order to remove the tag. Examples of suitable endoproteases for removal of a tag, include, for example, Factor Xa and TEV proteases.

(d) Other Targeting Moieties

In certain embodiments, the targeting moiety may comprise a receptor molecule, including, for example, receptors which naturally recognize a specific desired molecule of a target tissue. Such receptor molecules include receptors that have been modified to increase their specificity of interaction with a target molecule, receptors that have been modified to interact with a desired target molecule not naturally recognized by the receptor, and fragments of such receptors (see, e.g., Skerra, 2000, J. Molecular Recognition, 13:167-187). A preferred receptor is a chemokine receptor. Exemplary chemokine receptors have been described in, for example, Lapidot et al, 2002, Exp Hematol, 30:973-81 and Onuffer et al, 2002, Trends Pharmacol Sci, 23:459-67.

In other embodiments, the targeting moiety may comprise a ligand molecule, including, for example, ligands which naturally recognize a specific desired receptor of a target tissue. Such ligand molecules include ligands that have been modified to increase their specificity of interaction with a target receptor, ligands that have been modified to interact with a desired receptor not naturally recognized by the ligand, and fragments of such ligands.

In still other embodiments, the targeting moiety may comprise an aptamer. Aptamers are oligonucleotides that are selected to bind specifically to a desired molecular structure of the target tissue. Aptamers typically are the products of an affinity selection process similar to the affinity selection of phage display (also known as in vitro molecular evolution). The process involves performing several tandem iterations of affinity separation, e.g., using a solid support to which the desired immunogen is bound, followed by polymerase chain reaction (PCR) to amplify nucleic acids that bound to the immunogens. Each round of affinity separation thus enriches the nucleic acid population for molecules that successfully bind the desired immunogen. In this manner, a random pool of nucleic acids may be “educated” to yield aptamers that specifically bind target molecules. Aptamers typically are RNA, but may be DNA or analogs or derivatives thereof, such as, without limitation, peptide nucleic acids (PNAs) and phosphorothioate nucleic acids.

In yet other embodiments, the targeting moiety may be a peptidomimetic. By employing, for example, scanning mutagenesis to map the amino acid residues of a protein which is involved in binding other proteins, peptidomimetic compounds can be generated which mimic those residues which facilitate the interaction. Such mimetics may then be used as a targeting moiety to deliver a progenitor cell to a target tissue. For instance, non-hydrolyzable peptide analogs of such residues can be generated using benzodiazepine (e.g., see Freidinger et al. in Peptides: Chemistry and Biology, G. R. Marshall ed., ESCOM Publisher: Leiden, Netherlands, 1988), azepine (e.g., see Huffman et al. in Peptides: Chemistry and Biology, G. R. Marshall ed., ESCOM Publisher: Leiden, Netherlands, 1988), substituted gamma lactam rings (Garvey et al. in Peptides: Chemistry and Biology, G. R. Marshall ed., ESCOM Publisher: Leiden, Netherlands, 1988), keto-methylene pseudopeptides (Ewenson et al., 1986, J Med Chem 29:295; and Ewenson et al. in Peptides: Structure and Function (Proceedings of the 9th American Peptide Symposium) Pierce Chemical Co. Rockland, Ill., 1985), b-turn dipeptide cores (Nagai et al., 1985, Tetrahedron Lett 26:647; and Sato et al., 1986, J Chem Soc Perkin Trans 1:1231), and β-aminoalcohols (Gordon et al., 1985, Biochem Biophys Res Commun 126:419; and Dann et al., 1986, Biochem Biophys Res Commun 134:71).

6. Linkers and Lipophilic Moieties

In certain embodiments, a targeting moiety of the invention may be directly associated with a progenitor cell, This may be achieved, for example, by modifying the targeting moiety with a lipophilic moiety to allow insertion into or association with the cell membrane. Methods for inserting a palmitated antibody into a cell membrane are described, for example, in Colsky and Peacock, J Immunol Methods, 1989 124:179-87. Direct attachment to a cell may also be achieved by covalently attaching the targeting moiety to another element that has an affinity for a marker on the surface of the cell to be coated, such as an extracellular protein or oligosaccharide.

In other embodiments, a targeting moiety of the invention may be indirectly associated with a progenitor cell. Indirect attachment may be achieved, for example, by providing a linker that associates with the progenitor cell to be coated and with the targeting moiety. Exemplary linkers include Protein G. Protein G is a highly stable surface receptor from Streptococcus sp. (Lancefield Group G), that has four Fc-fragment binding sites for immunoglobulins and each molecule can bind 2 molecules of IgG (Bjorck L and G. 1984; Boyle and Reis 1987). Another exemplary linker is Protein A, which also binds Fc fragments, but with a different range of specificities. Linkers may be modified to associate with a progenitor cell through any of the various approaches described above with respect to direct attachment of a targeting moiety. For example, the linker may be modified with a lipophilic moiety. In certain exemplary embodiment, the linker is palmitated protein G or palmitated protein A.

There are a wide range of lipophilic moieties with which linkers or targeting moieties may be derivatived, including without limitation, palmitoyl moiety, myristoyl moiety, margaroyl moiety, stearoyl moiety, arachidoyl moiety, acetyl moiety, butytyl moiety, hexanoyl moiety, octanoyl moiety, decanoyl moiety, lauroyl moiety, palmitoleoyl moiety, behenoyl moiety, and lignoceroyl moiety. Preferred lipophilic moieties include palmitoyl moiety, myristoyl moiety, and margaroyl moiety. A lipophilic group can be, for example, a relatively long chain alkyl or cycloalkyl (preferably n-alkyl) group having approximately 7 to 30 carbons. The alkyl group may terminate with a hydroxy or primary amine “tail” To further illustrate, lipophilic molecules include alicyclic hydrocarbons, saturated and unsaturated fatty acids and other lipid and phospholipid moieties, waxes, cholesterol, isoprenoids, terpenes and polyalicyclic hydrocarbons including adamantane and buckminsterfullerenes, vitamins, polyethylene glycol or oligoethylene glycol, (C1-C18)-alkyl phosphate diesters, —O—CH2—CH(OH)—O—(C12-C18)-alkyl, conjugates with pyrene derivatives, esters and alcohols, other lipid molecules, cage structures such as adamantane and buckminsterfullerenes, and aromatic hydrocarbons such as benzene, perylene, phenanthrene, anthracene, naphthalene, pyrene, chrysene, and naphthacene.

Optionally, the lipophilic moiety can be a lipophilic dye suitable for use in the invention include, but are not limited to, diphenylhexatriene, Nile Red, N-phenyl-1-naphthylamine, Prodan, Laurodan, Pyrene, Perylene, rhodamine, rhodamine B, tetramethylrhodamine, Texas Red, sulforhodamine, 1,1′-didodecyl-3,3,3′,3′tetramethylindocarbocyanine perchlorate, octadecyl rhodamine B and the BODIPY dyes available from Molecular Probes Inc. Other exemplary lipophilic moieties include aliphatic carbonyl radical groups such as decanoyl, dodecanoyl, dodecenoyl, tetradecadienoyl, decynoyl, or dodecynoyl.

The N-terminal amine of a protein can be modified preferentially relative to other amines in a protein because its lower pKa results in higher amounts of the reactive unprotonated form at neutral or acidic pH. Aryl halides, aldehydes and ketones, acid anhydrides, isocyanates, isothiocyanates, imidoesters, acid halides, N-hydroxysuccinimidyl (e.g., sulfo-NHS-acetate), nitrophenyl esters, acylimidazoles, and other activated esters and thioesters are among those known to react with amine functions.

There are a variety of chemical methods for the modification of many amino acid side chains, such as cysteine, lysine, histidine, aspartic acid, glutamic acid, serine, threonine, tyrosine, arginine, methionine, and tryptophan. Therefore a lipophilic moiety may be attached to an amino acid other than at the N-terminus.

To illustrate, there are a large number of chemical cross-linking agents that are known to those skilled in the art. Heterobifunctional cross-linkers provide the ability to design more specific coupling methods for conjugating to proteins, thereby reducing the occurrences of unwanted side reactions such as homo-protein polymers. A wide variety of heterobifunctional cross-linkers are known in the art. These include: succinimidyl 4-(N-maleimidomethyl) cyclohexane-1-carboxylate (SMCC), m-Maleimidobenzoyl-N-hydroxysuccinimide ester (MBS); N-succinimidyl (4-iodoacetyl) aminobenzoate (SLAB), succinimidyl 4-(p-maleimidophenyl) butyrate (SMPB), 1-ethyl-3-(3-dimethylaminopropyl) carbodiimide hydrochloride (EDC); 4-succinimidyloxycarbonyl-a-methyl-a-(2-pyridyldithio)-tolune (SMPT), N-succinimidyl 3-(2-pyridyldithio) propionate (SPDP), succinimidyl 6-[3-(2-pyridyldithio) propionate] hexanoate (LC-SPDP). Those cross-linking agents having N-hydroxysuccinimide moieties can be obtained as the N-hydroxysulfosuccinimide analogs, which generally have greater water solubility. In addition, those cross-linking agents having disulfide bridges within the linking chain can be synthesized instead as the alkyl derivatives so as to reduce the amount of linker cleavage in vivo.

In addition to the heterobifunctional cross-linkers, there exists a number of other cross-linking agents including homobifunctional and photoreactive cross-linkers. Disuccinimidyl suberate (DSS), bismaleimidohexane (BMH) and dimethylpimelimidate.2 HCl (DMP) are examples of useful homobifunctional cross-linking agents, and bis-[β-(4-azidosalicylamido)ethyl]disulfide (BASED) and N-succinimidyl-6(4′-azido-2′-nitrophenyl-amino)hexanoate (SANPAH) are examples of useful photoreactive cross-linkers for use in this invention. For a recent review of protein coupling techniques, see Means et al. (1990), Bioconjugate Chemistry, 1:2-12, incorporated by reference herein.

One particularly useful class of heterobifunctional cross-linkers, included above, contain the primary amine reactive group, N-hydroxysuccinimide (NHS), or its water soluble analog N-hydroxysulfosuccinimide (sulfo-NHS). Primary amines (lysine epsilon groups) at alkaline pH's are unprotonated and react by nucleophilic attack on NHS or sulfo-NHS esters. This reaction results in the formation of an amide bond, and release of NHS or sulfo-NHS as a by-product.

In certain embodiments, the lipophilic moiety employed is a lipid moiety. Generally, a “lipid” is a member of a heterogeneous class of hydrophobic substances characterized by a variable solubility in organic solvents and insolubility, for the most part, in water. The principal class of lipids that are encompassed within this invention are fatty acids and sterols (e.g., cholesterol). Derivatized proteins of the invention contain fatty acids which are cyclic, acyclic (i.e., straight chain), saturated or unsaturated, mono-carboxylic acids. Exemplary saturated fatty acids have the generic formula: CH3(CH2)nCOOH. The following Table II lists examples of some fatty acids that can be derivatived conveniently using conventional chemical methods.

TABLE II
Exemplary Saturated and Unsaturated Fatty Acids.

| | |
|---|---|
| Value of n | Common Name |

Saturated Acids: CH3(CH2)nCOOH

| | |
|---|---|
| 2 | butyric acid |
| 4 | caproic acid |
| 6 | caprylic acid |
| 8 | capric acid |
| 10 | lauric acid |
| 12 | myristic acid |
| 14 | palmitic acid |
| 16 | stearic acid |
| 18 | arachidic acid |
| 20 | behenic acid |
| 22 | lignoceric acid |

Unsaturated Acids

| | |
|---|---|
| CH3CH═CHCOOH | crotonic acid |
| CH3(CH2)3CH═CH(CH2)7COOH | myristoleic acid |
| CH3(CH2)5CH═CH(CH2)7COOH | palmitoleic acid |
| CH3(CH2)7CH═CH(CH2)7COOH | oleic acid |
| CH3(CH2)3(CH2CH═CH)2(CH2)7COOH | linoleic acid |
| CH3(CH2CH═CH)3(CH2)7COOH | linolenic acid |
| CH3(CH2)3(CH2CH═CH)4(CH2)3COOH | arachidonic acid |

Other lipids that can be attached include branched-chain fatty acids and those of the phospholipid group such as the phosphatidylinositols (i.e., phosphatidylinositol 4-monophosphate and phosphatidylinositol 4,5-biphosphate), phosphatidycholine, phosphatidylethanolamine, phosphatidylserine, and isoprenoids such as farnesyl or geranyl groups.

7. Bioactive Factors

In certain aspects, compositions and methods of the present invention further comprise a bioactive factor, such as a growth factor, a cytokine or a chemokine. Such bioactive factors may regulate the growth, differentiation, and/or function of the progenitor cell. The bioactive factors may be added with the progenitor cell. Optionally, the bioactive factors may be added subsequent to the delivery of the progenitor cell.

To illustrate, the bioactive factor may be selected from a growth factor of the transforming growth factor β superfamily (e.g., a TGFβ or a TGFα); a bone morphogenetic protein (BMP, e.g., BMP2 or BMP4); cartilage-derived morphogenic proteins (CDMPs, e.g., CDMP-1 or CDMP-2) and growth differentiation factors (e.g., GDF5); angiogenic factors (e.g., angiogenin); platelet-derived cell growth factor (PD-ECGF); platelet-derived growth factors (PDGFs, e.g., PDGF-A, PDGF-B, and PDGF-BB); vascular endothelial growth factor (VEGF); a member of the epidermal growth factor family (e.g., EGF, TGFs, and PDGFs); fibroblast growth factors (e.g., bFGF); hepatocyte growth factors (HGFs); insulin-like growth factors (e.g., IGF-I and IGF-II); nerve growth factors (NGFs); colony-stimulating factor (e.g., CSF or GM-CSF); neurotrophin (e.g., NT-3, 4 or 5); growth hormones (GHs); interleukins (e.g., IL-1, IL-15); connective tissue growth factors (CTGFs); parathyroid hormone related proteins (PTHrp); chemokine; Wnt protein; Noggin; Gremlin; and mixtures of two or more of these factors.

8. Methods of Cell Delivery

In certain aspects, the present invention provides methods of delivering a progenitor cell to a target tissue in a subject. In certain embodiments, the method is a two-step approach, which comprises coating a progenitor cell with a linker and then contacting the coated progenitor cell with a targeting moiety that binds to both the linker and the target tissue. In other embodiments, the method is a one-step approach, which comprises directly coating the progenitor cell with a targeting moiety that binds to both the target tissue and the progenitor cell.

The progenitor cell having been either directly or indirectly complexed with the targeting moiety can be administered to a subject by a variety of means. Such administration methods, in view of this specification, are apparent to those of skill in the art. In certain embodiments, the progenitor cell is delivered to the subject by injection into blood. In other embodiments, the progenitor cell is delivered to the subject by injection into the target tissue. In still other embodiments, the progenitor cell is delivered to the subject by surgical implantation. In still other embodiments, the progenitor cell is delivered to the subject by subcutaneous injection. In yet other embodiments, the progenitor cell is delivered to the subject by intra-peritoneal injection. In yet other embodiments, the progenitor cell is delivered to the subject by intra-synovial injection.

In certain embodiments, progenitor cells may be inserted into a delivery device which facilitates introduction by injection or implantation into the subjects. Such delivery devices may include tubes or intraluminal devices, e.g. catheters, for injecting cells and fluids into the body of a recipient subject. In a preferred embodiment, the tubes additionally have a needle, e.g., a syringe, through which the cells of the invention can be introduced into the subject at a desired location.

The progenitor cells may be prepared for delivery in a variety of different forms. For example, the cells may be suspended in a solution or gel or embedded in a support matrix when contained in such a delivery device. Cells may be mixed with a pharmaceutically acceptable carrier or diluent in which the progenitor cells of the invention remain viable. Pharmaceutically acceptable carriers and diluents include saline, aqueous buffer solutions, solvents and/or dispersion media. The use of such carriers and diluents is well known in the art. The solution is preferably sterile and fluid. Preferably, the solution is stable under the conditions of manufacture and storage and preserved against the contaminating action of microorganisms such as bacteria and fungi through the use of, for example, parabens, chlorobutanol, phenol, ascorbic acid, thimerosal, and the like. Solutions of the invention may be prepared by incorporating cells as described herein in a pharmaceutically acceptable carrier or diluent and, as required, other ingredients enumerated above, followed by filtered sterilization.

9. Methods of Treating Diseases or Tissue Injuries

In certain aspects, the present invention provides methods of treating a disease or a tissue injury. For example, the tissue injury may result from laceration, burns, poison or extremes of temperature. Such methods comprise: a) providing a progenitor cell linked to a targeting moiety, wherein the targeting moiety selectively directs the progenitor cell to a diseased or injured target tissue; and b) delivering the progenitor cell linked with the targeting moiety to the diseased or injured target tissue. Optionally, the method of treating a disease/injury can be used alone or in combination with other therapies.

Progenitor cells derived from the embryo and from adult tissues have been shown to have extensive potentials for self-renewal and differentiation (see, e.g., Triffitt, 2002, J Cell Biochem, Suppl 38:13-9; Vats et al., 2002, Clin Otolaryngol., 27:227-32; Stocum, 2001, Wound Repair Regen, 9:429-42). Thus, a wide variety of diseases or injuries may be treated by delivering a progenitor cell to a target diseased or injured tissue so that the malfunctional target tissue can be specifically replaced with a functional tissue derived from the progenitor cell. Examples of diseases and injuries include without limitation, diabetes, cardiovascular disease, amyotrophic lateral sclerosis, Parkinson's disease, Huntington's disease, multiple sclerosis, stroke, myocardial infarction, spinal cord injury, brain injury, peripheral neuropathy, autoimmune diseases, liver based metabolic diseases, acute liver failure, chronic liver disease, leukemia, sickle-cell anemia, bone defects, muscular dystrophy, burns, osteoarthritis, and macular degeneration.

To illustrate, muscle stem cells have been shown to participate in regeneration after muscle damage and may be used for treating muscular dystrophy (see, e.g., Torrente et al., 2001, J Cell Biol, 152:335-48). Fetal neural cells, which are mixtures of multipotent neural stem cells, more restricted neural and glial precursors, and terminally differentiating cells, have been used successfully to reverse symptoms of Parkinson's and Huntington's diseases (see, e.g., Bjorklund et al, 2000, Nature Neurosci, 3:537-44). Hematopoietic stem cells, when injected into mouse myocardium infarcted by coronary artery ligation, can differentiate into proliferating cardiomyocytes and vascular structures, suggesting their use in treating cardiovascular diseases (see, e.g., Orlic et al., 2001, Nature, 410:701-5). Mesenchymal stem cells have been shown promise in the repair of cartilage, tendon, and segmental bone defects (see, e.g., Wakitani et al., 1994, J Bone Joint Surg, 76:579-92; Young et al., 1998, J Orthop Res, 16:406-13; Kadiyala et al., 1997, Tissue Eng, 3:173-85; Bruder et al., 1998, J Bone Joint Surg, 80:985-96; Bruder et al., 1998, J Orthop Res, 16:155-62). Transplanted neural stem cells were well integrated into the host's ischemic-injured retinas, suggesting their use in repairing retina (see, e.g., Kurimoto et al., 2001, Neurosci Lett, 306:57-60).

Exemplary progenitor cells and the related diseases or injuries have also been described in the following U.S. patents (prefaced by “US”) and international patent applications (prefaced by “WO”): U.S. Pat. No. 5,130,141; U.S. Pat. No. 5,786,217; U.S. Pat. No. 6,328,960; U.S. Pat. No. 6,387,369; WO 01/42425; WO 01/23528; WO 01/39784; WO 02/09650; WO 02/36829.

10. Tissue Engineering

In certain aspects, the present invention provides composition and methods of tissue engineering. Tissue engineering provides the opportunity to generate living substitutes for tissues and organs, which may overcome the drawbacks of classical tissue reconstruction.

In certain embodiments, the present invention provides a tissue engineering composition which comprises: a) a progenitor cell; b) a targeting moiety that binds to a target tissue; and c) a biocompatible scaffold. Such tissue engineering composition generates a scaffold graft to be delivered to a target tissue. Optionally, tissue engineering composition may generate a scaffold graft that can each include one type of progenitor cell or multiple types of progenitor cells.

In other embodiments, the present invention provides a method of delivering a scaffold graft in a target tissue, comprising: a) linking a progenitor cell to a targeting moiety that binds to a target tissue; b) seeding the progenitor cell from (a) onto a scaffold, thereby forming a scaffold graft; and c) implanting the scaffold graft from (b) in direct contact with, or adjacent to, a target tissue for a sufficient time, wherein cells of the target tissue associate with the implanted scaffold graft, thereby to form new tissue. For example, the scaffold graft can be delivered in a target tissue by surgical implantation. Optionally, such methods may further comprise removing the scaffold graft from the subject. For example, the scaffold graft removed from the subject (i.e., the scaffold and the tissue it bears at the end of the implantation period) can then be re-grafted into another target tissue. To illustrate, the scaffold graft removed from a tendon or ligament can then be re-grafted into a joint to repair a ruptured or otherwise damaged ligament.

As described herein, the biocompatible scaffold can consist of bioresorbable or non-bioresorbable materials. If the scaffold consists of a single bioresorbably material, it is preferably one that does not significantly resorb during the period of time when the target tissue is being laid down on or within it. Such scaffolds will generate a scaffold graft that includes living cells and essentially retain their shape and mechanical integrity. In some instances, it may be preferable to use scaffolds containing bioresorbable materials that lose, for example, less than a 2% of their weight during the same period. If the scaffold is constructed with two or more bioresorbable materials, it may be preferable to select the bioresorbable material that provides the scaffold with its structural integrity according to these criteria.

A wide range of bioresorbable materials is well known in the art, with varying in vivo resorption times. Moreover, the resorption time of a single material itself can also vary significantly with the molecular weight. By blending or copolymerizing different bioresorbable materials and/or by modifying the molecular weights of the materials, it is possible to tailor the resorption time of the bioresorbable material to the requirement at hand.

In certain embodiments, the bioresorbable materials for the biocompatible scaffold include bioresorbable polymers or copolymers that comprise the following monomers or mixtures of polymers and/or copolymers formed thereby: hydroxy acids, particularly lactic acid; glycolic acid; caprolactone; hydroxybutyrate; dioxanone; orthoesters; orthocarbonates; aminocarbonates.

Optionally, the bioresorbable materials can also include natural materials such as collagen, cellulose, fibrin, hyaluronic acid, fibronectin, chitosan, or mixtures of two or more of these materials. The bioresorbable materials may also comprise devitalized xenograft and/or devitalized allograft. Bioresorbable ceramics can also be included within the scaffold. Preferred bioresorbable materials include poly(lactic acid), poly(glycolic acid), polydioxanone, polyhydroxybutyrate, and poly(trimethylene carbonate), or mixtures thereof. Poly(lactic acid) has good mechanical strength and does not resorb quickly. Thus, its mechanical properties can be retained for a time sufficient for tissue in-growth to occur (at which point the tissue can assume some, if not all, of the load-bearing function of the scaffold (see A. G. A. Coombes and M. C. Meikle, “Resorbable Synthetic Polymers as Replacements for Bone Graft,” Clinical Materials, 17:35-67, 1994). Samples of poly(lactic acid) have been shown to lose only one or two percent of their weight over a 12-week trial.

In certain embodiments, the non-bioresorbable materials for the biocompatible scaffold include polyesters, particularly aromatic polyesters, such as polyalkylene terephthalates; polyamides; polyalkenes such as polyethylene and polypropylene; poly(vinyl fluoride), polytetrafluoroethylene carbon fibres; silk (natural or synthetic); carbon fibre; glass; and mixtures of these materials. An advantage of non-bioresorbable materials is that they essentially retain their initial mechanical properties. Thus, their strength does not lessen over time.

Preferably, the biocompatible scaffold is at least partially porous so that it allows tissue in-growth. When the scaffold contains interconnected pores that are evenly distributed, cells can infiltrate essentially all areas of the scaffold during the period of implantation. The pore diameter is determined by, in part, the need for adequate surface area for tissue in-growth and adequate space for nutrients and growth factors to reach the cells. In certain embodiments, the biocompatible scaffold may comprise a woven, non-woven (fibrous material), knitted, braided material, a foam, a sponge, a dendritic material, or a mixture of two or more of these. Optionally, the scaffold can be planar in form, cut or otherwise formed, if necessary, to an appropriate shape. For example, the scaffold can form a quadrilateral, circle, triangle, or other geometric shape in plan view.

In certain embodiments, the biocompatible scaffold can include certain additional components. For example, the scaffold may include bioactive factors, such as growth factors, cytokines or chemokines.

In other embodiments, hydrogels can also be included in the biocompatible scaffold. For example, the hydrogel can be incorporated within and/or around the scaffold prior to implantation to facilitate the transfer of cells and other biological material (e.g., growth factors) from the surrounding tissue into the scaffold. Hydrogels include positively charged, negatively charged, and neutral hydrogels, and can be either saturated or unsaturated. Examples of hydrogels are TETRONICS™ and POLOXAMINES™, which are poly(oxyethylene)-poly(oxypropylene) block copolymers of ethylene diamine; polysaccharides, chitosan, poly(vinyl amines), poly(vinyl pyridine), poly(vinyl imidazole), polyethylenimine, poly-L-lysine, growth factor binding or cell adhesion molecule binding derivatives, derivatized versions of the above (e.g., polyanions, polycations, peptides, polysaccharides, lipids, nucleic acids or blends, block-copolymers or combinations of the above or copolymers of the corresponding monomers); agarose, methylcellulose, hydroxyproylmethylcellulose, xyloglucan, acetan, carrageenan, xanthangum/locust beangum, gelatine, collagen (particularly Type 1), PLURONICS™, POLOXAMERS™, POLY(N-isopropylacrylmide), and N-isopropylacryhnide copolymers.

### EXEMPLIFICATION

The invention now being generally described, it will be more readily understood by reference to the following examples, which are included merely for purposes of illustration of certain aspects and embodiments of the present invention, and are not intended to limit the invention.

Rabbit Articular Chondrocytes

New Zealand rabbit articular chondrocytes were harvested as previously described (Wakitani et al., 1998, Tissue Eng., 4:429-44.) with minor alterations. Briefly, rabbit distal femoral condyles and proximal humeral condyles were harvested after the rabbits have been sacrificed by Fatal-Plus® (Vortech, Dearborn, Mich.) injection. The articular cartilage layer was scraped off the condyle using a scalpel, minced into 1 mm² pieces which were digested in a mixture of enzymes (Collagenase 1%, Trypsin 0.05% and Chondroitinase 0.1%) in Dulbecco's modified Eagle's Medium over night at 37° C. in 5% CO₂/95% air with constant gentle mixing. The mixture was filtered through a 70 μm filter to obtain a single cell suspension. The filtered solution was centrifuged at 300×g for five minutes and supernatant discarded and replaced with fresh Dulbecco's Modefied Eagle's Medium (DMEM) supplemented with 10% selected lots (Lennon, et al., 1995, Exp Cell Res., 219:211-22) of fetal calf serum (FCS, Gibco BRL, Gaithersburg, Md.) and antibiotic-antimycotic solution (Penicillin G sodium: 100 U/ml, Amphotericin B: 0.5 μg/ml, streptomycin sulfate: 100 μg/ml; Gibco/BRL). The cells were counted with a hemocytometer and plated in 100 mm Petri-dishes at 2.0×10⁵ cells per plate. The first medium change is done 48-72 hours after plating after which the medium is changed twice a week.

Palmitation of Protein G

Recombinant protein G (Sigma, St. Louis, Mo.) was derivatized with N-hydroxysuccinimide ester of palmitic acid (Sigma, St. Louis, Mo.) using the procedure described by Kim and Peacock (Kim, et al., 1993, J Immunol Methods, 158:57-65) for palmitation of protein A. The lipid-derivatized protein G was purified on a 10 ml Sephadex G-25 (Pharmacia, Piscataway, N.J.) column equilibrated with PBS containing 0.1% deoxycholate (DOC) pH 7.4. The protein concentration was adjusted to 750 μg/ml by O.D. absorbance (UV-160 spectrophotometer, Shimadru) at 280 nm according to standard curves, 20 μm filter sterilized, and stored at 4° C. until used.

Membrane Incorporation of Palmitated Protein G and the Effects on Cell Viability and Mitotic Potential.

In vitro expanded chondrocytes were trypsinized off the plates, washed three times in serum free DMEM and re-suspended at a density of 3-4×10⁶/ml in DMEM. Varying concentrations of palmitated protein G (PPG) or non-derivatized protein G (as a negative control) were added to the cell suspension, and the mixture was incubated at 37° C. for 2 hours with constant gentle mixing. To assess the incorporation of PPG onto cell surfaces, the cells were washed twice in the buffer (PBS, 0.1% DOC pH 7.4) and then incubated at 4° C. for 1 hour with 1001 μl of 100 μg/ml of FITC-human IgG (Sigma) diluted in the same buffer (per 1.0×10⁶ cells). Cells were washed three times in the buffer and analyzed at the Flow Cytometry Core Facility at Case Western Reserve University (National Cancer Institute Core Facility, Cleveland, Ohio, U.S.A.) by fluorescent microscopy. The toxicity of rising concentrations of PPG coating was assessed using propidium iodine uptake as quantified by FACS scan. An aliquot of cells from every concentration was re-plated on 100 mm petri-dishes in complete medium allowed to attach and incubated at 37° C. in 5% CO₂/95% air. The cells were trypsinized after one week incubation, counted by a hemocytometer to determine the effects of PPG coating on cell growth.

Aggregate Cultures

Aggregate cultures (Yoo et al., 1998, J Bone Joint Surg Am., 80:1745-57) were used to assess chondrogenic potential of antibody-coated cells. Cells were coated with a range of coating concentrations of PPG (0-60 μg/ml) and a second coating with human FITC IgG antibody. Cells were placed in 0.5 ml of defined medium (Dulbecco's Modified Eagle medium base supplemented with 6.25 μg/ml insulin, 6.26 μg/ml transferrin, 6.25 μg/ml selenious acid, 5.35 μg/ml linoleic acid, 1.25 μg/ml bovine serum albumin (BSA), 1 mM pyruvate, and 37.5 ng/ml ascorbate-2-phospate) 2.0×10⁵ cells per 15 ml polypropylene conical tube and centrifuged at 500×g for five minutes. The pellets were incubated at 37° C. in 5% CO₂/95% air, for three weeks with medium changes every other day. Within the first 24 hours, the cells formed a free-floating pellet. At three weeks, the pellets were harvested and fixed in 10% neutral buffered formalin for standard histology. The chondrogenic phenotype was assessed by examination of histologic sections stained with toluidine blue (chondrogenic cells are round, surrounded by a meta-chromatic staining representing highly sulfated glycosaminoglycans). In order to further verify the phenotype of the cells within the aggregates, type II collagen immunohistochemistry staining was carried out as previously described (Naumann, et al., 2002, J Histochem Cytochem., 50:1049-58). Briefly, sections were rehydrated with PBS for 5 minutes, and digested with bovine testis Hyalruronidase 8000 U/ml (Sigma H-3506) for 60 minutes. A second digestion was performed using Pronase 1 mg/ml (Sigma P-5147) for 15 minutes at 20° C. after which non-specific adhesion sites were blocked using 3% BSA. Next, the sections were stained with mouse anti-collagen type II IgG (II-116B3) diluted in 3% BSA 1:200 for 60 minutes. The slides are washed with 3% BSA and coated with second layer of horseradish peroxidase-conjugate goat-anti mouse IgG. Slides were washed in PBS and contrasted in a solution of Vector VIP Substrate (Vector labs; Burlin-game, CA) according to the manufacturers instructions, washed and counterstained with fast green. The slides were observed on an Olympus BH-2 fluorescence microscope.

Cell Coating with Matrix Specific Antibodies.

Cells pre-coated with PPG were washed twice in buffer and then incubated at 4° C. for 1 hour with 100 μl of 100 μg/ml cartilage matrix specific antibodies diluted in the same buffer (per 1.0×10⁶ cells). The cartilage matrix specific antibodies used were: mouse anti-chondroitin 4 sulfate IgG (2B6), mouse anti-chondroitin 6 sulfate IgG (3B3; Seikasaku Kogyo, Inc., Tokyo), mouse anti-keratan sulfate IgG (5D4) (Caterson, Iowa City, Iowa). After this initial incubation the cells were washed twice in the same buffer and incubated for 1 hour with FITC-conjugated F(ab)′2 goat anti-mouse antibody (Sigma) diluted in the same buffer. After this last incubation the cells were washed and the efficiency of coating was assessed by FACS.

Vybrant™ Staining of Cells

One day prior to coating of the cells with PPG, the cells were incubated in 10 μM Vybrant™ (Molecular Probes, Eugene, Oreg.) in Hank's balanced salt solution for 15 minutes at 37° C. in 5% CO₂/95% air after which they were washed once with Hank's balanced salt solution and fresh medium was added. This vital staining of cells is based on the passive diffusion of a colorless, nonfluorescent carboxy-fluorescein diacetate succinimidyl ester (CFDA SE) into cells. Once in the cell, the CFDA SE is cleaved by intracellular esterases to yield a highly fluorescent dye which is retained in some cells for a number of weeks. Staining of the cells was verified by fluorescent microscopy after trypsinization of the cells and before the PPG coating procedure.

Frozen Sections

5-8 μm frozen sections of rabbit articular cartilage were cut and placed onto 3-amino propyltriethoxysilane coated slides (Sigma) and stored at −20° C. until use. When tested the slides were first hydrated in PBS for 30 minutes. Half the sections were incubated in chondroitinase ABC (0.1 U/ml) for 15 minutes, and all sections were then blocked with 1% BSA/PBS for 5 minutes. Next, the sections were incubated for 45 minutes with 30 μl of 10×10⁶ Vybrant™ stained cells in PBS coated with different antibodies (or PPG as a negative control). After the incubation time the sections are gently and meticulously washed with PBS for 5 minutes, suspended for 30 seconds in 5 μg/ml propidium iodine, washed again, and cover-slipped with 1 mg/ml p-phenylendiamine in 45% glycerol in 1 N sodium phosphate, pH 8.5. The slides were analyzed using fluorescent microscopy.

Osteo-Chondral Explants

Osteo-Chondral explants were harvested from 1-year-old male New Zealand white rabbits after they were sacrificed by intra-venous phenobarbital overdose (2,600 mg/kg; Fetal-Plus, Vortex Pharmaceuticals, Dearborn, Mich.). The distal femoral condyles were sterilely harvested and 4.25 mm diameter trephine is used to manually harvest 3-4 osteo-chondral cylinders from every femur. A standard defect is then created by sliding a 1 mm diameter ring curette along the cartilage surface, this is performed taking care as to not penetrate the subchondral bone. These explants were incubated in a 96-well plate with the cartilage side facing up and the different Vybrant™ stained cells (1.5×10⁶ cells/well) coated with the different antibodies are applied to the well on top of the explants and incubated for 45 minutes at 37° C. in 5% CO₂/95% air. Following this incubation, the explants were turned cartilage side facing down into empty wells filled with DMEM. Using a conical insert, the cartilage is kept above the bottom of the well thus allowing gravity to affect the attached cells. This incubation was carried out for 12 hours. The explants are then harvested, fixed in 10% neutral buffered formalin, decalcified, embedded, and analyzed by fluorescent microscopy.

Membrane Incorporation of Palmitated Protein G and the Effects on Cell Viability and Mitotic Potential

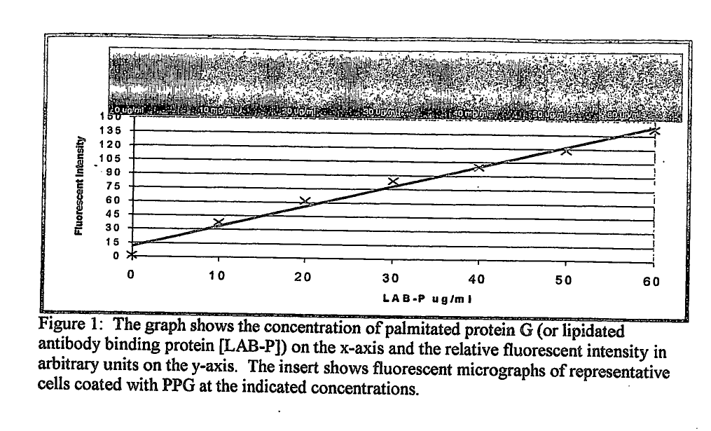

A two-step strategy was developed for quantitatively coating cells with cartilage matrix specific antibodies. The first set of optimization procedures is aimed at efficiently coating cells with PPG. In order to coat cells with antibodies from different species, it was necessary to test the use of PPG in the coating procedure. To test the ability of PPG to coat cells, cells were incubated in a range of PPG concentrations and as a negative control, cells incubated with buffer only or with non-palmitated protein G. Cells incubated with buffer only or with non-palmitated protein G did not bind significant amounts of FITC labeled human IgG (FIG. 1). A linear increase of mean fluorescence intensity was observed in samples incubated in 10-60 μg/ml of PPG (FIG. 1). To verify coating of the cells with the second layer of matrix specific antibodies (2B6, 3B3, 5D4 and II-116B3), cells incubated in primary antibodies were washed and incubated with goat anti-mouse FITC labeled antibody (F(ab′)₂ fragment). After washing the cells twice in buffer, fluorescence was quantified by FACS (results not shown). The results showed that PPG coated cells were, in fact, coated with matrix specific antibodies.

Effects of Coating with PPG on Cell Viability, Mitotic Potential and Chondrogenic Phenotype.

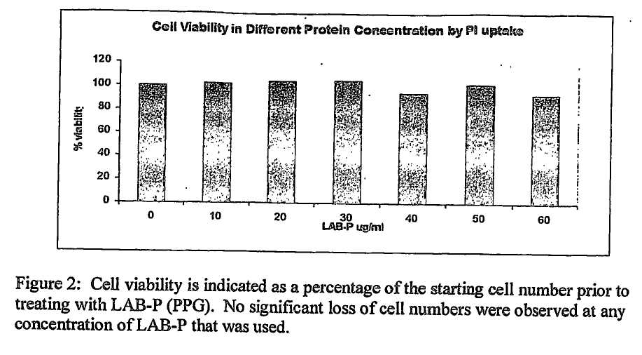

Propidium iodine uptake, assessed by FACS, was used to assess the effects of the coating procedure on cellular viability. The results (FIG. 2) showed above 95% viability of cells coated with concentrations of up to 60 μg/ml PPG.

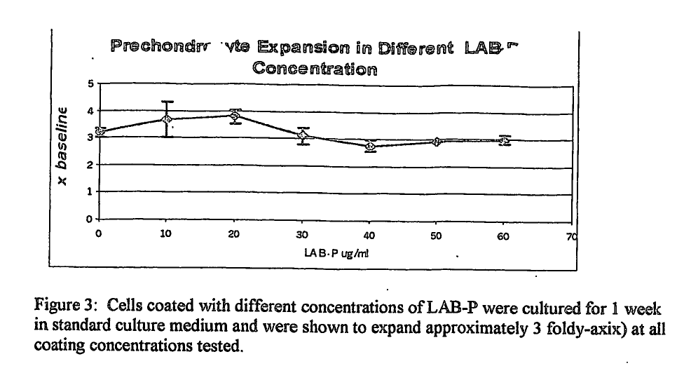

Mitotic expansion of PPG cells was analyzed by incubating identical number of cells (2.0×10⁵) coated with different concentration of PPG in 100 mm petri-dishes. After 1 week of incubation at 37° C. in 5% CO₂/95% air the cells were trypsinized and counted. These results showed no adverse effect of cell painting on mitotic expansion. PPG coated cells tripled in number in all PPG concentrations tested (10-60 μg/ml) and no significant differences were observed between PPG samples and uncoated controls (FIG. 3).

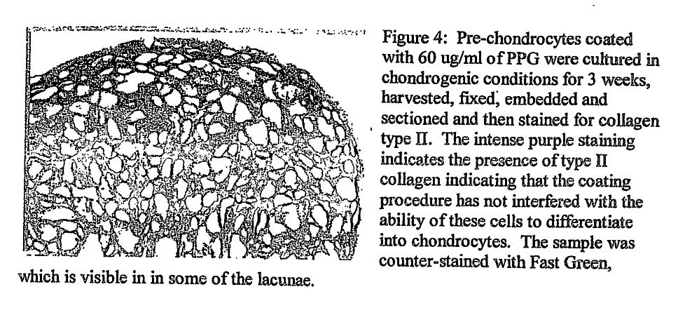

Cells coated with PPG-FITC labeled human IgG formed oval aggregates after 1 week in culture in chondrogenic culture conditions, and generally grew in size by 3 weeks in culture. Histologic examination of toluidine blue-stained 5-μm sections of three week old aggregates showed rounded cells surrounded by abundant meta-chromatic stained matrix indicating a high sulfated glycosaminoglycan content (FIG. 4), which correlates with cartilage matrix. To confirm the chondrocyte phenotype in these samples, sections were assayed by immunohistochemistry for expression of collagen type II, and this analysis revealed the presence of collagen type II plus cell matrix (data not shown).

Targeting Frozen Sections

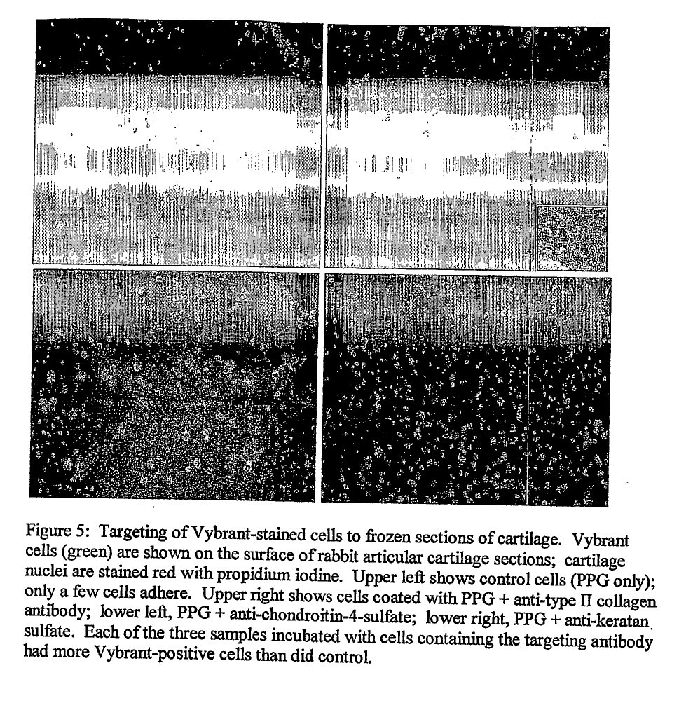

The chondrocytes were first incubated in a vital dye, Vybrant™, which is metabolized into the fluorescent molecule only by living cells. Once the cells were stained they were coated with PPG and a second layer of matrix specific antibodies. Fluorescent micrographs showed that cells coated with specific matrix antibodies are found in greater density on the sections than in controls (FIG. 5).

Osteo-Chondral Explants

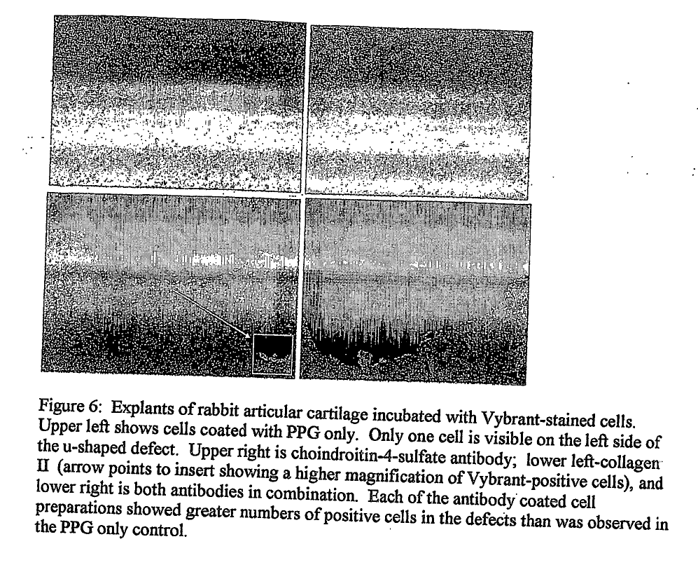

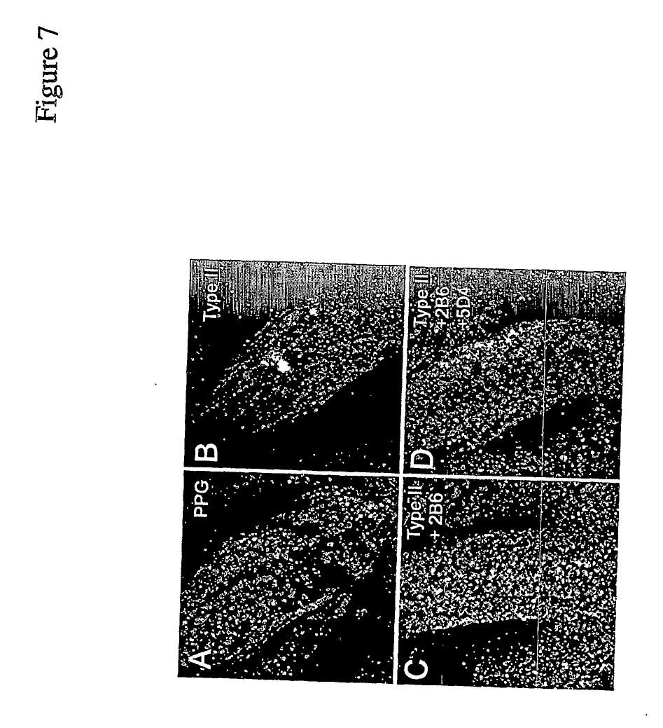

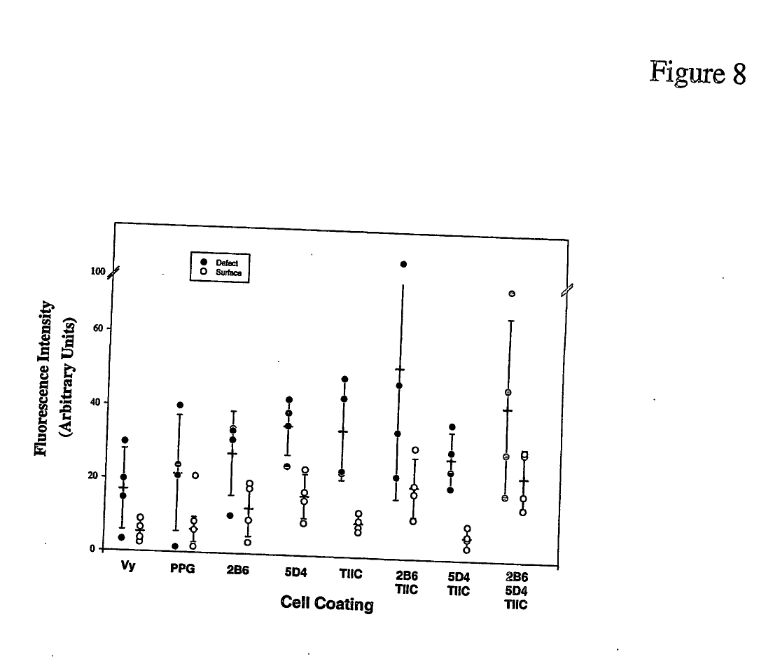

To test the ability of antibody-coated cells to preferentially bind to cartilage matrix, Vybrant™ labeled cells were used in order to assess the targeting potential of our antibody coated cells. A system was developed to allow us to create a standard articular defect in an osteochondral explant. Fluorescent micrograph revealed greater number of cells preferentially inside the defect than on the native cartilage surface when specific antibodies were used and a different morphology of the cells inside the defect. Cells that adhered inside the defect without specific antibody coating had a flattened appearance while specifically targeted cells seem to be round and clumped in groups. It also appears that combining the different antibodies together in the coating of cells has an additive effect (FIGS. 6, 7 and 8).

Incorporation by Reference

All publications and patents mentioned herein are hereby incorporated by reference in their entirety as if each individual publication or patent was specifically and individually indicated to be incorporated by reference. In case of conflict, the present application, including any definitions herein, will control.

Equivalents

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A cell delivery composition comprising:
a progenitor cell; and
a targeting moiety that binds to a target tissue,
wherein said targeting moiety selectively directs the progenitor cell to the target tissue.

2. The composition of claim 1, wherein said progenitor cell is selected from the group consisting of a totipotent stem cell, pluripotent stem cell, multipotent stem cell, mesenchymal stem cell, neuronal stem cell, hematopoietic stem cell, pancreatic stem cell, cardiac stem cell, embryonic stem cell, embryonic germ cell, neural crest stem cell, kidney stem cell, hepatic stem cell, lung stem cell, hemangioblast cell, and endothelial progenitor cell.

3. The composition of claim 1, wherein said progenitor cell is derived from a de-differentiated chondrogenic cell, myogenic cell, osteogenic cell, tendogenic cell, ligamentogenic cell, adipogenic cell, and dermatogenic cell.

4. The composition of claim 1, wherein said progenitor cell is pre-coated with a linker selected from the group consisting of protein G and protein A.

5. (canceled)

6. The composition of claim 4, wherein said linker is modified with a lipophilic moiety selected from a palmitoyl moiety, myristoyl moiety, margaroyl moiety, stearoyl moiety, arachidoyl moiety, acetyl moiety, butytyl moiety, hexanoyl moiety, octanoyl moiety, decanoyl moiety, lauroyl moiety, palmitoleoyl moiety, behenoyl moiety, and lignoceroyl moiety.

7. (canceled)

8. The composition of claim 1, wherein said progenitor cell is directly linked to said targeting moiety.

9. The composition of claim 8, wherein said targeting moiety is modified with a lipophilic moiety selected from a palmitoyl moiety, myristoyl moiety, margaroyl moiety, stearoyl moiety, arachidoyl moiety, acetyl moiety, butytyl moiety, hexanoyl moiety, octanoyl moiety, decanoyl moiety, lauroyl moiety, palmitoleoyl moiety, behenoyl moiety, and lignoceroyl moiety.

10. (canceled)

11. The composition of claim 1, wherein said progenitor cell expresses a cell surface marker or an extracellular matrix molecule selected from the group consisting of CD4, CD8, CD10, CD30, CD33, CD34, CD38, CD45, CD133, CD146, fetal liver kinase-1 (Flk1), C-kit, Lin, Mac-1, Sca-1, Stro-1, Thy-1, Collagen types II or IV, O1, O4, N-CAM, p75, and SSEA.

12. (canceled)

13. The composition of claim 1, wherein said targeting moiety comprises a component of a specific binding pair.

14. The composition of claim 1, wherein said targeting moiety interacts with an epitope intrinsic to the target tissue.

15. The composition of claim 14, wherein said epitope is a protein or carbohydrate epitope of the target tissue.

16. The composition of claim 15, wherein said carbohydrate epitope is within a complex carbohydrate.

17. The composition of claim 16, wherein said complex carbohydrate binds to a lectin.

18. The composition of claim 16, wherein said complex carbohydrate is a proteoglycan selected from the group consisting of chondroitin sulfate, dermatan sulfate, heparin, heparan sulfate, hyaluronate, and keratan sulfate.

19. (canceled)

20. The composition of claim 1, wherein said targeting moiety comprises a homing peptide.

21. The composition of claim 20, wherein said homing peptide comprises a sequence selected from PWERSL, FMLRDR, and SGLRQR, and targets to bone marrow tissues.

22. The composition of claim 20, wherein said homing peptide comprises a sequence of ASSLNIA, and targets to muscle tissues.

23. The composition of claim 20, wherein said homing peptide comprises a sequence of YSGKWGW, and targets to the intestine.

24. The composition of claim 20, wherein said homing peptide comprises a sequence selected from CGFELETC and CGFECVRQCPERC, and targets to lung tissues.

25. The composition of claim 20, wherein said homing peptide selectively directs the progenitor cell to the target tissue.

26. The composition of claim 1, wherein said targeting moiety comprises an antibody selected from antibodies to collagens I, II, V, VI and IX, chondroitin-4-sulfate, dermatan sulfate, and chondroitin-6-sulfate.

27-28. (canceled)

29. The composition of claim 26, wherein said antibody is selected from a monoclonal antibody, a polyclonal antibody, and a humanized antibody.

30-31. (canceled)

32. The composition of claim 1, wherein said targeting moiety is a fusion protein.

33. The composition of claim 32, wherein said fusion protein comprises an Fc fragment.

34. The composition of claim 32, wherein said fusion protein comprises a homing peptide.

35. The composition of claim 1, wherein said targeting moiety comprises a receptor or ligand.

36. The composition of claim 35, wherein said receptor is a chemokine receptor.

37. The composition of claim 1, wherein said targeting moiety comprises an aptamer.

38. The composition of claim 1, wherein said targeting moiety is a peptidomimetic.

39. The composition of claim 1, wherein the target tissue is selected from cartilage, skeletal muscle, smooth muscle, bone, tendon, ligament, adipose tissue, skin, neuronal tissue, connective tissue, hepatic tissue, pancreatic tissue, kidney tissue, bone marrow tissue, cardiac tissue, retinal tissue, intestinal tissue, lung tissue, and endothelium tissue.

40. (canceled)

41. The composition of claim 1, said composition further comprising a bioactive factor selected from a transforming growth factor, a bone morphogenic protein, a cartilage-derived morphogenic protein, a growth differentiation factor, an angiogenic factor, a platelet-derived growth factor, a vascular endothelial growth factor, an epidermal growth factor, a fibroblast growth factor, a hepatocyte growth factor, an insulin-like growth factor, a nerve growth factor, a colony-stimulating factor, a neurotropin, a growth hormone, an interleukin, a connective tissue growth factor, a parathyroid hormone-related protein, a chemokine, a Wnt protein, a Noggin, and a Gremlin.

42-44. (canceled)

45. The method of claim 190, wherein the progenitor cell is selected from the group consisting of a totipotent stem cell, pluripotent stem cell, multipotent stem cell, mesenchymal stem cell, neuronal stem cell, hematopoietic stem cell, pancreatic stem cell, cardiac stem cell, embryonic stem cell, embryonic germ cell, neural crest stem cell, kidney stem cell, hepatic stem cell, lung stem cell, hemangioblast cell, and endothelial progenitor cell.

46. The method of claim 190, wherein the progenitor cell is derived from a de-differentiated chondrogenic cell, myogenic cell, osteogenic cell, tendogenic cell, ligamentogenic cell, adipogenic cell, and dermatogenic cell.

47. The method of claim 190, wherein the linker is selected from protein G and protein A.

48. The method of claim 190, wherein the linker is modified with a lipophilic moiety selected from a palmitoyl moiety, myristoyl moiety, margaroyl moiety, stearoyl moiety, arachidoyl moiety, acetyl moiety, butytyl moiety, hexanoyl moiety, octanoyl moiety, decanoyl moiety, lauroyl moiety, palmitoleoyl moiety, behenoyl moiety, and lignoceroyl moiety.

49. (canceled)

50. The method of claim 191, wherein the targeting moiety is modified with a lipophilic moiety selected from a palmitoyl moiety, myristoyl moiety, margaroyl moiety, stearoyl moiety, arachidoyl moiety, acetyl moiety, butytyl moiety, hexanoyl moiety, octanoyl moiety, decanoyl moiety, lauroyl moiety, palmitoleoyl moiety, behenoyl moiety, and lignoceroyl moiety.

51. (canceled)

52. The method of claim 190, wherein the progenitor cell expresses a cell surface marker or an extracellular matrix molecule selected from the group consisting of CD4, CD8, CD10, CD30, CD33, CD34, CD38, CD45, CD133, CD146, fetal liver kinase-1 (Flk1), C-kit, Lin, Mac-1, Sca-1, Stro-1, Thy-1, Collagen types II or IV, O1, O4, N-CAM, p75, and SSEA.

53. (canceled)

54. The method of claim 190, wherein the targeting moiety comprises a component of a binding pair.

55. The method of claim 190, wherein the targeting moiety interacts with an epitope intrinsic to the target tissue.

56. The method of claim 55, wherein the epitope is a protein or carbohydrate epitope of the target tissue.

57. The method of claim 56, wherein the carbohydrate epitope is within a complex carbohydrate.

58. The method of claim 57, wherein the complex carbohydrate binds to a lectin.

59. The method of claim 57, wherein the complex carbohydrate is a proteoglycan selected from the group consisting of chondroitin sulfate, dermatan sulfate, heparin, heparin sulfate, hyaluronate, and keratin sulfate.

60. (canceled)

61. The method of claim 190, wherein the targeting moiety comprises a homing peptide.

62. The method of claim 61, wherein the homing peptide comprises a sequence selected from PWERSL, FMLRDR, and SGLRQR, and targets to bone marrow tissues.

63. The method of claim 61, wherein the homing peptide comprises a sequence of ASSLNIA, and targets to muscle tissues.

64. The method of claim 61, wherein the homing peptide comprises a sequence of YSGKWGW, and targets to the intestine.

65. The method of claim 61, wherein the homing peptide comprises a sequence selected from CGFELETC and CGFECVRQCPERC, and targets to lung tissues.

66. The method of claim 61, wherein the homing peptide selectively directs the progenitor cell to the target tissue.

67. The method of claim 190, wherein the targeting moiety comprises an antibody selected from antibodies to collagens I, II, V, VI and IX, chondroitin-4-sulfate, dermatan sulfate, and chondroitin-6-sulfate.

68-69. (canceled)

70. The method of claim 67, wherein the antibody is selected from a monoclonal antibody, a polyclonal antibody, and a humanized antibody.

71-72. (canceled)

73. The method of claim 190, wherein the targeting moiety is a fusion protein.

74. The method of claim 73, wherein the fusion protein comprises an Fc fragment.

75. The method of claim 73, wherein the fusion protein comprises a homing peptide.

76. The method of claim 190, wherein the targeting moiety comprises a receptor or ligand.

77. The method of claim 76, wherein the receptor is a chemokine receptor.

78. The method of claim 190, wherein the targeting moiety comprises an aptamer.

79. The method of claim 190, wherein the targeting moiety is a peptidomimetic.

80. The method of claim 190, wherein the target tissue is selected from cartilage, skeletal muscle, smooth muscle, bone, tendon, ligament, adipose tissue, skin, neuronal tissue, connective tissue, hepatic tissue, pancreatic tissue, kidney tissue, bone marrow tissue, cardiac tissue, retinal tissue, intestinal tissue, lung tissue, and endothelium tissue.

81. (canceled)

82. The method of claim 190, wherein the progenitor cell is treated with a bioactive factor selected from a transforming growth factor, a bone morphogenic protein, a cartilage-derived morphogenic protein, a growth differentiation factor, an angiogenic factor, a platelet-derived growth factor, a vascular endothelial growth factor, an epidermal growth factor, a fibroblast growth factor, a hepatocyte growth factor, an insulin-like growth factor, a nerve growth factor, a colony-stimulating factor, a neurotropin, a growth hormone, an interleukin, a connective tissue growth factor, a parathyroid hormone-related protein, a chemokine, a Wnt protein, a Noggin, and a Gremlin.

83. (canceled)

84. The method of claim 190, wherein the progenitor cell complexed with the targeting moiety is delivered to the subject by injection into blood.

85. The method of claim 190, wherein the progenitor cell complexed with the targeting moiety is delivered to the subject by injection into the target tissue.

86. The method of claim 190, wherein the progenitor cell complexed with the targeting moiety is delivered to the subject by surgical implantation.

87. The method of claim 190, wherein the progenitor cell complexed with the targeting moiety is delivered to the subject by subcutaneous injection.

88. The method of claim 190, wherein the progenitor cell complexed with the targeting moiety is delivered to the subject by intra-peritoneal injection.

89. The method of claim 190, wherein the progenitor cell complexed with the targeting moiety is delivered to the subject by intra-synovial injection.

90-95. (canceled)

96. A tissue engineering composition, comprising:
a progenitor cell;
a targeting moiety that binds to a target tissue; and
a biocompatible scaffold,
wherein the tissue engineering composition generates a scaffold graft to be delivered to a target tissue.

97. The composition of claim 96, wherein said scaffold comprises a bioresorbable material having at least one molecule selected from a hydroxy acid, a glycolic acid, caprolactone, hydroxybutyrate, dioxanone, an orthoester, an orthocarbonate, an aminocarbonate, collagen, cellulose, fibrin, hyaluronic acid, fibronectin, and chitosan.

98. (canceled)

99. The composition of claim 96, wherein said scaffold comprises a non-bioresorbable material having at least one molecule selected from a polyalkylene terephthalate, a polyamide, a polyalkene, poly(vinyl fluoride), polytetrafluoroethylene carbon fibers, natural or synthetic silk, carbon fiber, and glass.

100. (canceled)

101. The composition of of claim 96, further comprising a bioactive factor selected from a transforming growth factor, a bone morphogenic protein, a cartilage-derived morphogenic protein, a growth differentiation factor, an angiogenic factor, a platelet-derived growth factor, a vascular endothelial growth factor, an epidermal growth factor, a fibroblast growth factor, a hepatocyte growth factor, an insulin-like growth factor, a nerve growth factor, a colony-stimulating factor, a neurotropin, a growth hormone, an interleukin, a connective tissue growth factor, a parathyroid hormone-related protein, a chemokine, a Wnt protein, a Noggin, and a Gremlin.

102. (canceled)

103. The composition of claim 96, wherein said progenitor cell is selected from the group consisting of a totipotent stem cell, pluripotent stem cell, multipotent stem cell, mesenchymal stem cell, neuronal stem cell, hematopoietic stem cell, pancreatic stem cell, cardiac stem cell, embryonic stem cell, embryonic germ cell, neural crest stem cell, kidney stem cell, hepatic stem cell, lung stem cell, hemangioblast cell, and endothelial progenitor cell.

104. The composition of claim 96, wherein said progenitor cell is derived from a de-differentiated chondrogenic cell, myogenic cell, osteogenic cell, tendogenic cell, ligamentogenic cell, adipogenic cell, and dermatogenic cell.

105. The composition of claim 96, wherein said progenitor cell is pre-coated with a linker selected from protein G and protein A.

106. (canceled)

107. The composition of claim 105, wherein said linker is modified with a lipophilic moiety selected from a palmitoyl moiety, myristoyl moiety, margaroyl moiety, stearoyl moiety, arachidoyl moiety, acetyl moiety, butytyl moiety, hexanoyl moiety, octanoyl moiety, decanoyl moiety, lauroyl moiety, palmitoleoyl moiety, behenoyl moiety, and lignoceroyl moiety.

108. (canceled)

109. The composition of claim 96, wherein said progenitor cell is directly linked to said targeting moiety.

110. The composition of claim 109, wherein said targeting moiety is modified with a lipophilic moiety selected from a palmitoyl moiety, myristoyl moiety, margaroyl moiety, stearoyl moiety, arachidoyl moiety, acetyl moiety, butytyl moiety, hexanoyl moiety, octanoyl moiety, decanoyl moiety, lauroyl moiety, palmitoleoyl moiety, behenoyl moiety, and lignoceroyl moiety.

111. (canceled)

112. The composition of claim 96, wherein said progenitor cell expresses a cell surface marker or an extracellular matrix molecule selected from the group consisting of CD4, CD8, CD10, CD30, CD33, CD34, CD38, CD45, CD133, CD146, fetal liver kinase-1 (Flk1), C-kit, Lin, Mac-1, Sca-1, Stro-1, ThV-1, Collagen types II or IV, O1, O4, N-CAM, p75, and SSEA.

113. (canceled)

114. The composition of claim 96, wherein said targeting moiety comprises a component of a specific binding pair.

115. The composition of claim 96, wherein said targeting moiety interacts with an epitope intrinsic to the target tissue.

116. The composition of claim 115, wherein said epitope is a protein or carbohydrate epitope of the target tissue.

117. The composition of claim 116, wherein said carbohydrate epitope is within a complex carbohydrate.

118. The composition of claim 117, wherein said complex carbohydrate binds to a lectin.

119. The composition of claim 117, wherein said complex carbohydrate is a proteoglycan selected from the group consisting of chondroitin sulfate, dermatan sulfate, heparin, heparan sulfate, hyaluronate, and keratan sulfate.

120. (canceled)

121. The composition of claim 96, wherein said targeting moiety comprises a homing peptide.

122. The composition of claim 121, wherein said homing peptide comprises a sequence selected from PWERSL, FMLRDR, and SGLRQR, and targets to bone marrow tissues.

123. The composition of claim 122, wherein said homing peptide comprises a sequence of ASSLNIA, and targets to muscle tissues.

124. The composition of claim 121, wherein said homing peptide comprises a sequence of YSGKWGW, and targets to the intestine.

125. The composition of claim 121, wherein said homing peptide comprises a sequence selected from CGFELETC and CGFECVRQCPERC, and targets to lung tissues.

126. The composition of claim 121, wherein said homing peptide selectively directs the progenitor cell to the target tissue.

127. The composition of claim 96, wherein said targeting moiety comprises an antibody selected from antibodies to collagens I, II, V, VI and IX, chondroitin-4-sulfate, dermatan sulfate, and chondroitin-6-sulfate.

128-129. (canceled)

130. The composition of claim 127, wherein said antibody is selected from a monoclonal antibody, a polyclonal antibody, and a humanized antibody.

131-132. (canceled)

133. The composition of claim 96, wherein said targeting moiety is a fusion protein.

134. The composition of claim 133, wherein said fusion protein comprises an Fc fragment.

135. The composition of claim 133, wherein said fusion protein comprises a homing peptide.

136. The composition of claim 96, wherein said targeting moiety comprises a receptor or ligand.

137. The composition of claim 136, wherein said receptor is a chemokine receptor.

138. The composition of claim 96 wherein said targeting moiety comprises an aptamer.

139. The composition of claim 96, wherein said targeting moiety is a peptidomimetic.

140. The composition of claim 96, wherein the target tissue is selected from cartilage, skeletal muscle, smooth muscle, bone, tendon, ligament, adipose tissue, skin, neuronal tissue, connective tissue, hepatic tissue, pancreatic tissue, kidney tissue, bone marrow tissue, cardiac tissue, retinal tissue, intestinal tissue, lung tissue, and endothelium tissue.

141. (canceled)

142. A method of delivering a scaffold graft in target tissue, comprising:
linking a progenitor cell to a targeting moiety that binds to a target tissue;
seeding the progenitor cell onto a biocompatible scaffold, thereby forming a scaffold graft; and
implanting the scaffold in direct contact with, or adjacent to, a target tissue for a sufficient time,
wherein cells of the target tissue associate with the implanted scaffold graft, thereby to form new tissue.

143. The method of claim 142, wherein the scaffold comprises a bioresorbable material having at least one molecule selected from a hydroxy acid, a glycolic acid, caprolactone, hydroxybutyrate, dioxanone, an orthoester, an orthocarbonate, an aminocarbonate, collagen, cellulose, fibrin, hyaluronic acid, fibronectin, and chitosan.

144. (canceled)

145. The method of claim 142, wherein the scaffold comprises a non-bioresorbable material having at least one molecule selected from a polyalkylene terephthalate, a polyamide, a polyalkene, poly(vinyl fluoride), polytetrafluoroethylene carbon fibers, natural or synthetic silk, carbon fiber, and glass.

146. (canceled)

147. The method of claim 142, wherein the progenitor cell is selected from the group consisting of a totipotent stem cell, pluripotent stem cell, multipotent stem cell, mesenchymal stem cell, neuronal stem cell, hematopoietic stem cell, pancreatic stem cell, cardiac stem cell, embryonic stem cell, embryonic germ cell, neural crest stem cell, kidney stem cell, hepatic stem cell, lung stem cell, hemangioblast cell, and endothelial progenitor cell.

148. The method of claim 142, wherein the progenitor cell is derived from a de-differentiated chondrogenic cell, myogenic cell, osteogenic cell, tendogenic cell, ligamentogenic cell, adipogenic cell, and dermatogenic cell.

149. The method of claim 142, wherein the progenitor cell is pre-coated with a linker selected from protein G and protein A.

150. (canceled)

151. The method of claim 149, wherein the linker is modified with a lipophilic moiety selected from a palmitoyl moiety, myristoyl moiety, margaroyl moiety, stearoyl moiety, arachidoyl moiety, acetyl moiety, butytyl moiety, hexanoyl moiety, octanoyl moiety, decanoyl moiety, lauroyl moiety, palmitoleoyl moiety, behenoyl moiety, and lignoceroyl moiety.

152. (canceled)

153. The method of claim 142, wherein the progenitor cell is directly linked to said targeting moiety.

154. The method of claim 153, wherein the targeting moiety is modified with a lipophilic moiety selected from a palmitoyl moiety, myristoyl moiety, margaroyl moiety, stearoyl moiety, arachidoyl moiety, acetyl moiety, butytyl moiety, hexanoyl moiety, octanoyl moiety, decanoyl moiety, lauroyl moiety, palmitoleoyl moiety, behenoyl moiety, and lignoceroyl moiety.

155. (canceled)

156. The method of claim 142, wherein the progenitor cell expresses a cell surface marker or an extracellular matrix molecule selected from the group consisting of CD4, CD8, CD10, CD30, CD33, CD34, CD38, CD45, CD133, CD146, fetal liver kinase-1 (Flk1), C-kit, Lin, Mac-1, Sca-1, Stro-1, Thy-1, Collagen types II or IV, O1, O4, N-CAM, p75, and SSEA.

157. (canceled)

158. The method of claim 142, wherein the targeting moiety comprises a component of a specific binding pair.

159. The method of claim 142, wherein the targeting moiety interacts with an epitope intrinsic to the target tissue.

160. The method of claim 159, wherein the epitope is a protein or carbohydrate epitope of the target tissue.

161. The method of claim 160, wherein the carbohydrate epitope is within a complex carbohydrate.

162. The method of claim 161, wherein the complex carbohydrate binds to a lectin.

163. The method of claim 161, wherein the complex carbohydrate is a proteoglycan selected from the group consisting of chondroitin sulfate, dermatan sulfate, heparin, heparan sulfate, hyaluronate, and keratan sulfate.

164. (canceled)

165. The method of claim 142, wherein the targeting moiety comprises a homing peptide.

166. The method of claim 165, wherein the homing peptide comprises a sequence selected from PWERSL, FMLRDR, and SGLRQR, and targets to bone marrow tissues.

167. The method of claim 165, wherein the homing peptide comprises a sequence of ASSLNIA, and targets to muscle tissues.

168. The method of claim 165, wherein the homing peptide comprises a sequence of YSGKWGW, and targets to the intestine.

169. The method of claim 165, wherein the homing peptide comprises a sequence selected from CGFELETC and CGFECVRQCPERC, and targets to lung tissues.

170. The method of claim 165, wherein the homing peptide selectively directs the progenitor cell to the target tissue.

171. The method of claim 142, wherein the targeting moiety comprises an antibody selected from antibodies to collagens I, II, V, VI and IX, chondroitin-4-sulfate, dermatan sulfate, and chondroitin-6-sulfate.

172-173. (canceled)

174. The method of claim 171, wherein the antibody is selected from a monoclonal antibody, a polyclonal antibody, and a humanized antibody.

175-176. (canceled)

177. The method of claim 142, wherein the targeting moiety is a fusion protein.

178. The method of claim 177, wherein the fusion protein comprises an Fc fragment.

179. The method of claim 177, wherein the fusion protein comprises a homing peptide.

180. The method of claim 142, wherein the targeting moiety comprises a receptor or ligand.

181. The method of claim 180, wherein the receptor is a chemokine receptor.

182. The method of claim 142, wherein the targeting moiety comprises an aptamer.

183. The method of claim 142, wherein the targeting moiety is a peptidomimetic.

184. The method of claim 142, wherein the target tissue is selected from cartilage, skeletal muscle, smooth muscle, bone, tendon, ligament, adipose tissue, skin, neuronal tissue, connective tissue, hepatic tissue, pancreatic tissue, kidney tissue, bone marrow tissue, cardiac tissue, retinal tissue, intestinal tissue, lung tissue, and endothelium tissue.

185. (canceled)

186. The method of claim 142, wherein the scaffold graft comprises a bioactive factor selected from a transforming growth factor, a bone morphogenic protein, a cartilage-derived morphogenic protein, a growth differentiation factor, an angiogenic factor, a platelet-derived growth factor, a vascular endothelial growth factor, an epidermal growth factor, a fibroblast growth factor, a hepatocyte growth factor, an insulin-like growth factor, a nerve growth factor, a colony-stimulating factor, a neurotropin, a growth hormone, an interleukin, a connective tissue growth factor, a parathyroid hormone-related protein, a chemokine, a Wnt protein, a Noggin, and a Gremlin.

187. (canceled)

188. The method of claim 142, wherein the scaffold graft is delivered to the target tissue by surgical implantation.

189. The method of claim 142, further comprising removing the scaffold graft from the subject.

190. A method of delivering a progenitor cell to a target tissue in a subject, comprising:
coating the progenitor cell with a targeting moiety that binds to a target tissue and the progenitor cell; and
administering the progenitor cell complexed with the targeting moiety to a subject,
wherein said targeting moiety selectively directs the progenitor cell to the target tissue.

191. The method of claim 190, wherein said step of coating the progenitor cell further comprises the steps of:
coating the progenitor cell with a linker; and
contacting the coated progenitor cell with the targeting moiety so the targeting moiety binds to the linker and can then bind to the target tissue.
